(19) United States
(12) Patent Application Publication
(10) Pub. No.: US 2022/0362163 A1
(43) Pub. Date: Nov. 17, 2022

(54) METHODS AND COMPOSITIONS FOR CANCER TREATMENT USING NANOPARTICLES CONJUGATED WITH MULTIPLE LIGANDS FOR BINDING RECEPTORS ON NK CELLS

(71) Applicant: The University of North Carolina at Chapel Hill, Chapel Hill, NC (US)

(72) Inventors: Andrew Wang, Durham, NC (US); Kin Man Au, Carrboro, NC (US)

(21) Appl. No.: 17/769,917

(22) PCT Filed: Oct. 15, 2020
(86) PCT No.: PCT/US2020/055818

§ 371 Date: Apr. 18, 2022

Related U.S. Application Data
(60) Provisional application No. 62/923,060, filed on Oct. 18, 2019.

(51) Int. Cl. A61K 9/51; A61K 31/704; A61K 39/00; A61P 35/00

(52) U.S. Cl. CPC A61K 9/51; A61K 31/704; A61K 39/001104; A61P 35/00

(57) ABSTRACT

The present invention provides methods and compositions comprising a particle comprising at least one first targeting agent which binds a first target on an NK cell surface, and at least one second targeting agent which binds a second target on a cancer cell surface, wherein the second targeting agent is different from the first targeting agent.

### STATEMENT OF PRIORITY

This application claims the benefit, under 35 U.S.C. § 119(e), of U.S. Provisional Application No. 62/923,060, filed on Oct. 18, 2019, the entire contents of which are incorporated by reference herein.

### STATEMENT OF GOVERNMENT SUPPORT

This invention was made with government support under Grant Number CA198999 awarded by the National Institutes of Health. The government has certain rights in the invention.

### FIELD OF THE INVENTION

The present invention relates to immunoregulation and cancer immunotherapy.

### BACKGROUND OF THE INVENTION

Cancer immunotherapy, the utilization of the patient's own immune system to treat cancer, has emerged as a powerful new strategy in cancer treatment. Although most of the advances in cancer immunotherapy are focused on utilizing the adaptive immune system to eradicate cancer, there is a growing interest in harnessing the power of the innate immune response to shape anti-tumor immunity. Early correlative research has demonstrated that among the mechanisms of resistance to the adaptive immune system, tumor cells can evade the adaptive immune system through mutations that render the adaptive immune system ineffective. The key actor in the innate immune system is the Natural Killer (NK) cell, which serves as a first-line defense. Unlike adaptive immune cells (e.g., T- and B-cells), NK cells show spontaneous cytolytic activity against cancer cells without the need for neoantigens.

NK cell activation often involves the activation of more than one co-stimulatory molecule (e.g., CD16 and 4-1BB), and NK cell-mediated anticancer immunity is often hampered by the poor expression of NK cell-activating ligands and the overexpression of MHC I and other co-inhibitory molecules on the cancer cells. In recent years, several bispecific antibodies targeting NK cells and tumor cells have been successfully engineered to facilitate engagement and cytotoxicity, but their translation is hindered by on-target, off-tumor adverse events. More importantly, these bispecifics only contain one NK activating ligand, thus limiting NK activation.

The present invention overcomes shortcomings in the art by providing nanoparticles with at least one first targeting agent that binds a target on an NK cell surface, and at least one second target agent that binds a target on a cancer cell surface, and methods of using the same.

### SUMMARY OF THE INVENTION

The present invention provides methods and compositions for treating cancer, inducing cytotoxicity in a cancer cell, inducing an NK cell immune response, activating NK cells, and/or delivering a therapeutic agent to a cancer cell.

Thus, one aspect of the present invention provides a nanoparticle comprising: at least one first targeting agent that binds a first target on an NK cell surface; and at least one second targeting agent that binds a second target on a cancer cell surface, wherein the second targeting agent is different from the first targeting agent.

Further provided herein is a composition (e.g., a pharmaceutical composition) comprising a nanoparticle of the invention.

A further aspect of the present invention provides a method of activating an NK cell, comprising contacting the NK cell with the nanoparticle and/or composition of the present invention under conditions whereby the first targeting agent binds the first target on the NK cell surface.

Another aspect of the present invention provides a method of inducing an NK cell immune response, comprising contacting the NK cell with the nanoparticle and/or composition of the present invention under conditions whereby the first targeting agent binds the first target on the NK cell surface.

An additional aspect of the present invention provides a method of inducing cytotoxicity in a cancer cell, comprising contacting the cancer cell with the nanoparticle and/or composition of the present invention under conditions whereby the second targeting agent binds the second target on the surface of the cancer cell.

A further aspect of the present invention provides a method of delivering a therapeutic agent to a cancer cell, comprising contacting the cancer cell with the nanoparticle and/or composition of the present invention comprising a therapeutic agent under conditions whereby the second targeting agent binds the second target on the surface of the cancer cell, thereby delivering the therapeutic agent to the cancer cell.

A further aspect of the present invention provides a method of inducing an NK cell immune response in a subject in need thereof, comprising administering to the subject an effective amount of the nanoparticle and/or composition of the present invention under conditions whereby the first targeting agent binds the first target on the surface of the NK cell.

A further aspect of the present invention provides a method of activating NK cells in a subject in need thereof, comprising administering to the subject an effective amount of the nanoparticle and/or composition of the present invention under conditions whereby the first targeting agent binds the first target on the surface of the NK cell.

A further aspect of the present invention provides a method of treating cancer in a subject in need thereof, comprising administering to the subject an effective amount of the nanoparticle and/or composition of the present invention under conditions whereby the second targeting agent binds the second target on the surface of the cancer cell.

A further aspect of the present invention provides a method of treating cancer in a subject in need thereof, comprising administering to the subject an effective amount of the nanoparticle and/or composition of the present invention under conditions whereby the first targeting agent binds the first target on the surface of the NK cell and whereby the second agent binds the second target on the surface of the cancer cell.

A further aspect of the present invention provides a method of delivering a therapeutic agent to a cancer cell in a subject in need thereof, comprising administering to the subject an effective amount of the nanoparticle and/or composition of the present invention, wherein the nanoparticle and/or composition comprises a therapeutic agent, under conditions whereby the second targeting agent binds the second target on the surface of the cancer cell, thereby delivering the therapeutic agent to the cancer cell in the subject.

Additionally provided herein are kits comprising the nanoparticle and/or composition of the present invention; methods of use of the nanoparticle and/or composition of the present invention in activating NK cells, inducing cytotoxicity in a cancer cell, delivering a therapeutic agent to a cancer cell, and/or treating cancer; and preparations of a medicament for use comprising a particle and/or the composition of the present invention.

These and other aspects of the invention are addressed in more detail in the description of the invention set forth below.

### BRIEF DESCRIPTION OF THE DRAWINGS

FIGS. 1A-1D show the mechanism of action and characterization of EGFR-targeted trivalent nanoengagers. FIG. 1A shows a cartoon illustration of the mechanism of action of the EGFR-targeted trivalent nanoengagers against EGFR-overexpressed cancer after systemic administration. FIGS. 1B-1C show representative transmission electron microscope (TEM) images (FIG. 1B) and number-average particle (D_{N}) distribution curves (FIG. 1C) of EGFR-targeted drug-free and EPI-encapsulated nanoengagers (α-EGFR/α-CD16/α-4-1BB NPs). FIG. 1D shows pH-dependent in vitro drug release kinetics of antibody-free EPI NPs and α-EGFR/α-CD16/α-4-1BB EPI NPs (n=3).

FIG. 2 shows representative TEM images of different drug-free and EPI-encapsulated NPs.

FIG. 3 shows number-average particle diameter (D_{N}) distribution curves of different drug-free and EPI-encapsulated NPs determined by nanoparticle-tracking analysis (NTA) on nanoparticle dispersions in 0.1M phosphate buffered saline (PBS). All measurements were based on the average of five separate measurements.

FIGS. 4A-4B show in vitro drug release kinetics of non-targeted EPI NPs, α-EGFR EPI NPs, and α-EGFR/α-CD16/α-4-1BB EPI NPs under different physiological conditions. FIG. 4A shows time-dependent UV-visible spectra recorded for non-functionalized and different antibody-functionalized EPI-encapsulated NPs after being incubated in a large excess amount of 0.1 M PBS at pH 6.0 and 7.0 (at 37° C.). The nanoparticle concentration was 2 mg/mL. All measurements were based on the average of three separate measurements. FIG. 4B shows in vitro drug release profiles of non-targeted and different EGFR-targeted EPI NPs.

FIGS. 5A-5F show physicochemical properties of EGFR-targeted trifunctionalized nanoengagers. FIGS. 5A-5B show representative CLSM images (FIG. 5A) and FACS histograms (FIG. 5B) of CD3⁻ CD49b⁺ expanded murine NK cells after incubation with FITC-labeled (i) α-CD16 NPs, (ii) α-4-1BB NPs, (iii) α-EGFR NPs, (iv) α-CD16/α-4-1BB NPs, and (v) α-EGFR/α-CD16/α-4-1BB NPs. FIG. 5C shows representative FACS histograms of EGFR-overexpressed HT29, MB468, and A431 cells after incubation with FITC-labeled α-EGFR NPs, α-CD16/α-4-1BB NPs, and α-EGFR/α-CD16/α-4-1BB NPs (n=3). FIG. 5D shows binding affinities of different antibody-functionalized FITC-labeled NPs to the EGFR-negative Raji cells, as quantified by FACS. FIG. 5E shows direct in vitro toxicities of free EPI, non-targeted EPI NPs, and different antibody-functionalized EPI NPs against (i) HT29, (ii) MB468, and (iii) A431 cells, as assessed by MTS assay 3 days after initial treatment. FIG. 5F shows DNA damage induced by different EPI treatments. Representative FACS histograms of α-γ-H2AX (PE-labeled)-stained HT29, MB468, and A431 cells after being treated with 500 nM of different EPI formulations for 1 h. Treated cells were washed before, cultured for another 24 h, and then subjected to FACS. To quantify the DNA damage, treated cells were fixed and permeabilized before being stained with PE-labeled α-γ-H2AX.

FIGS. 6A-6H show that nanoengagers activate NK cells to attack cancer cells in vitro. FIG. 6A shows in vitro cytotoxicities of NK cells pretreated with α-CD16, α-4-1BB, α-CD16 NPs, α-4-1BB NPs, and their 1:1 combinations, and α-CD16/α-4-1BB NPs. The effector cells to target cells (E/T) ratio was 1:1. The cytotoxicities were determined 24 h after treatment. Data are presented as mean±SEM (n=6). Statistical significances were calculated by 2-way ANOVA followed by Tukey's HSD post-hoc test. *p<0.05. FIG. 6B shows quantification of bioluminescence of (non-irradiated) B16F10-Luc cells after being co-cultured with antibody-pretreated NK cells for 24 h. NK cells were pre-treated with free or NP-conjugated α-CD16 and/or α-4-1BB (at a concentration of 1 μg of each antibody per 1×10⁶ NK cell) at 37° C. for 30 min and washed once before being co-cultured with the seeded B16F10-Luc cells (2×10⁴ cells per well) at a 1:1 effector/target ratio. Live cells show strong bioluminescence signals after being incubated with Bright-Glo™ luciferase reagent (n=6). FIG. 6C shows quantification of bioluminescence of irradiated B16F10-Luc cells after co-culture with antibody-pretreated NK cells for 24 h. The B16F10-Luc cells were subjected to a 5 Gy cesium-137 irradiation 3 h before being co-cultured with antibody pre-treated NK cells. NK cells were pre-treated with free or NP-conjugated α-CD16 and/or α-4-1BB at a concentration of 1 μg of each antibody per 1×10⁶NK cell at 37° C. for 30 min, washed once before being co-cultured with the seeded B16F10-Luc cells (2×10⁴ cells per well) at a 1:1 effector/target ratio. Viable cells show strong bioluminescence signals after being incubated with Bright-Glo™ luciferase reagent (n=6). FIG. 6D shows representative phase-sensitive optical images of non-irradiated and 5 Gy irradiated B16F10 cells after incubation with NK cells pretreated with α-CD16 and α-4-1BB, α-CD16 NPs, α-4-1BB NPs, and α-CD16/α-4-1BB NPs. The E/T ratio was 1:1. Unbound NK cells were removed by washing before imaging. FIG. 6E shows in vitro cytotoxicities of NK cells against HT29-Luc2 cells that were pretreated with α-CD16, α-4-1BB, α-CD16/α-4-1BB NPs (with or without free α-EGFR or α-EGFR NPs), α-EGFR/α-CD16/α-4-1BB NPs (with/without free EPI or EPI NPs), and α-EGFR/α-CD16/α-4-1BB EPI NPs. The cytotoxicities were quantified 24 h after the treatment. The E/T ratio was 1:1. Data are presented as mean±SEM (n=6). Statistical significances were calculated by 2-way ANOVA followed by Tukey's HSD post hoc test. *p<0.05. FIG. 6F shows quantification of bioluminescence of HT29-Luc2 cells after being treated with different immunotherapeutics before being co-cultured with expanded NK cells at E/T=1:1. Left panel: Bioluminescence of HT29-Luc2 cells after being treated with different immunotherapeutics for 1 h, washed, before being further cultured in completed medium (in the absence of NK cells) for three days. The immunotherapeutic doses were 10 ng of each antibody per 1×10⁴ cells (in each well) with or without co-treatment with 600 nM of free/encapsulated EPI. The cell viabilities were quantified by Bright-Glo™ luciferase reagent. Live cells show strong bioluminescence signals after being incubated with the luciferase assay (n=6)). Right panel: Bioluminescence image of HT29-Luc2 cells after being treated with different immunotherapeutics for 1 h, washed, before being co-cultured with NK cells at an E/T=1:1 for three days. The immunotherapeutic doses were 10 ng of each antibody per 1×10⁴ cells (in each well) with or without co-treatment with 600 nM of free/encapsulated EPI. The cell viabilities were quantified by Bright-Glo™ luciferase reagent. Live cells show strong bioluminescence signals after being incubated with the luciferase assay (n=6). FIG. 6G shows viabilities of HT29, MB468, and A431 cells recorded three days after being treated with drug-free or EPI encapsulated α-EGFR/α-CD16/α-4-1BB NPs (containing 600 nM of encapsulated EPI or the same amount of drug-free NPs) in the presence or absence of NK cells (at 1:1 effector/target ratio). Data are presented as mean±SEM (n=8). Statistical significances were calculated by 2-way ANOVA followed by Tukey's HSD post-hoc test. *p<0.05. FIG. 6H shows viabilities of HT29, MB468, and A431 cells pretreated with sub-therapeutic doses of EPI-encapsulated trifunctionalized nanoengagers (α-EGFR/α-CD16/α-4-1BB NPs) before co-culture with NK cells. Cells (1×10⁴ cells per well for the MB468 and HT29 cells, or 5×10³ cells per well for the A431 cells) were treated with 1.2 μg of drug-free or EPI-encapsulated α-EGFR/α-CD16/α-4-1BB NPs for 1 h, washed, and then co-cultured with NK cells at an E/T=1:1 ratio. The absorbance (at 490 nm) of HT29 (panel a), MB468 (panel b) and A431 (panel c) cells after the addition of MTS assay. The NK cells alone showed insignificant absorbance at 490 nm because the cells lose viability in the cytokine-free culture media. Thus, the absorbance (at 490 nm) of the treatment groups co-cultured with NK cells should not be affected by the NK cells.

FIG. 7A-7C show spatiotemporal co-activation of CD16 and 4-1BB co-stimulatory molecules on NK cells delays murine tumor growth in vivo. In the immune cell-depleted C57BL/6 mouse model, B cells, NK cells, CD4⁺ T cells, and CD8⁺ T cells were depleted by intraperitoneal (i.p.) injection of α-CD20, α-NK1.1, α-CD4, and α-CD8 (300 μg/injection) at 5, 8, 10, 12, 15, and 18 days post-inoculation. Mouse IgG 2a (300 μg/injection) was administered as an isotype control. α-CD16/α-4-1BB NPs (containing 100 μg of each antibody) were intravenously (i.v.) administered at 6, 7, and 8 days post-inoculation. Immunotherapeutics containing 100 μg of α-CD16 and/or 100 μg of α-4-1BB (free or NP-conjugated) were tail vein i.v. administered at 6, 7, and 8 days post-inoculation. The xenograft tumors of mice in the immunostimulation groups were subjected to a single 5 Gy irradiation 4 h before the administration of immunotherapeutics to upregulate the NK cell-activating ligands in the cancer cells. FIGS. 7A-7B show average tumor growth curves (i), (ii), and survival curves (iii), (iv), of B16F10 tumor-bearing mice after receiving treatments with different immunotherapeutics (n=6 mice/group). FIG. 7C shows average tumor growth curves (1), and survival curves (ii) of B16F10 tumor-bearing immune cell-depleted mice after receiving treatments with α-CD16/α-4-1BB NPs (n=7 mice/group). Data are presented as mean±SEM. *p<0.05. Statistical significances of tumor growth curves were calculated via one-way ANOVA with a Tukey post-hoc test. *p<0.05. Statistical significances of survival curves were calculated via log-rank (Mantel-Cox) test. *p<0.05.

FIG. 8A shows spatiotemporal co-activation of CD16 and 4-1BB receptors delays non-irradiated B16F10 xenograft tumor growth in vivo. Lines show individual tumor growth curves of B16F10 tumor-bearing mice after receiving treatments with different immunotherapeutics without irradiation (n=6 mice/group).

FIG. 8B shows that 5 Gy of cesium-137 irradiation synergistically improves the anticancer activities of α-CD16/α-4-1BB in the B16F10 tumor model in C57BL/6 mice. FIG. 8B top panel: average tumor curves of non-irradiated and irradiated B16F10 tumor-bearing mice after different treatments with α-CD16/α-4-1BB NPs. At day 19 post-inoculation, 5 Gy IR before the first treatment with α-CD16/α-4-1BB NPs reduced the average tumor volume by about 60% compared with those that received 5 Gy IR. In contrast, treatment with α-CD16/α-4-1BB NPs (without IR) reduced the average tumor volume only by about 40% compared with the non-treatment group. This indicates that the 5 Gy IR synergistically (but not additively) improved the anticancer activities of α-CD16/α-4-1BB NPs. FIG. 8B bottom panel: survival curves of non-irradiated and irradiated B16F10 tumor-bearing mice after different treatments with α-CD16/α-4-1BB NPs. The average survival time for the non-treatment group was 19±1 days. Treatment with α-CD16/α-4-1BB NPs (without immunostimulation) increased the survival by an average of 3 days (i.e., average survival time=22±2 days). 5 Gy irradiation without further treatment increased the survival by an average of 3 days (i.e., average survival time=22±1 days). 5 Gy IR followed by α-CD16/α-4-1BB NPs treatment increased the survival time by 14 days (i.e., average survival time=32±1 days). This indicates the irradiation synergistically increased the anticancer activities of the α-CD16/α-4-1BB NPs.

FIG. 8C shows spatiotemporal co-activation of CD16 and 4-1BB receptors delays irradiated B16F10 xenograft tumor growth in vivo. Lines show individual tumor growth curves of B16F10 tumor-bearing mice after receiving treatments with different immunotherapeutics with 5 Gy irradiation before the first treatment (n=6 mice/group). FIG. 8D shows that α-CD16/α-4-1BB NPs effectively co-activated NK cells and improve cancer immunotherapy in vivo. Lines show individual tumor growth curves of immune cell-depleted B16F10 tumor-bearing mice after receiving treatments with α-CD16/α-4-1BB NPs or just received 5 Gy IR. (n=7 mice/group).

FIG. 8E shows anticancer activities of free α-CD16 plus free α-4-1BB and α-CD16/α-4-1BB NPs against B16F10 tumor in T cell-deficient Nu mice. FIG. 8E panel A shows an experimental scheme for a B16F10 tumor model in T cell-deficient Nu mice. Three doses of immunotherapeutics with 100 μg of each antibody were administered via tail vein injection at day 6, 7, and 8 post-inoculation. Mice in the immunostimulation groups received a single 5 Gy cesium-137 irradiation (IR) 3 h before the first administration of the immunotherapeutics. The in vivo efficacy study was terminated 21 days post-inoculation. Individual tumor growth curves (FIG. 8E panel B) and average tumor growth curves (FIG. 8E panel C) of B16F10 tumor-bearing mice recorded after treatment with different immunotherapeutics. Tumor growth inhibitions (TGIs) were calculated based on the average tumor volumes of treatment groups, and the non-treatment control group recorded at day 19 post-inoculation. Without immunostimulation (5 Gy IR), neither α-CD16 plus α-4-1BB nor α-CD16/α-4-1BB NPs inhibited tumor growth (p=0.6423 versus the non-treatment group). 5 Gy IR delayed tumor growth with a TGI of 36%. 5 Gy IR followed by treatment with α-CD16 plus α-4-1BB effectively delayed tumor growth with a TGI of 61%. 5 Gy IR followed by treatment with α-CD16/α-4-1BB NPs further delayed tumor growth with a TGI of 78% (p=0.0181 versus treatment with 5 Gy IR followed by α-CD16 plus α-4-1BB). n=6 mice for all groups. Statistical significance was calculated via one-way ANOVA with a Tukey post-hoc test.

FIGS. 9A-9D show that EGFR-targeted nanoengagers effectively inhibit EGFR-overexpressed tumor growth in vivo. FIG. 9A shows an experimental scheme for A431 and MB468 tumor models in T-cell deficient Nu mice. Three doses of immunotherapeutics/chemo-immunotherapeutics containing 100 μg of α-EGFR, 100 μg of α-CD16 and 100 μg of α-4-1BB (with/without 160 μg of free/encapsulated EPI) were tail vein i.v. administered at 6, 8, and 10 days post-inoculation. FIG. 9B shows average tumor growth curves (i), survival curves, and median survival (MS) (ii) recorded for A431 bearing Nu mice after receiving different treatments (n=6 mice/group). FIG. 9C shows average tumor growth curves and tumor growth inhibition (TGI) recorded for MB468 xenograft tumor-bearing Nu mice after receiving different treatments (n=6 mice/group). TGIs were calculated by comparing the average tumor volume change in the treatment groups related to the non-treatment group at the study endpoint (125 days post-inoculation). FIG. 9D shows an experimental scheme for EGFR⁺ HT29 and EGFR⁻ Raji dual-xenograft tumor model in T cell-deficient Nu mice. Three doses of immunotherapeutics/chemo-immunotherapeutics were tail vein i.v. administered at 6, 8, and 10 days post-inoculation. The in vivo efficacy study was terminated 20 days post-inoculation, when the large diameter of Raji tumor reached 10 mm. Average tumor growth curves of HT29 (i), and Raji (ii) xenograft tumors after receiving different treatments (n=5 for the non-treatment control group, n=7 for all other treatment groups). TGIs were calculated by comparing the average tumor volume change in the treatment groups related to the non-treatment group at the study endpoint (20 days post-inoculation). Data are presented as mean±SEM. Statistical significances average tumor growth curves were calculated via one-way ANOVA with a Tukey post-hoc test. *p<0.05. Statistical significances survival curves were calculated via the log-rank (Mantel-Cox) test. *p<0.05.

FIG. 10A shows that EGFR-targeted nanoengagers (α-EGFR/α-CD16/α-4-1BB NPs) effectively inhibit the growth of EGFR-overexpressed A431 tumors in vivo. Lines show individual tumor growth curves recorded for A431 bearing Nu mice after receiving different treatments (n=6 mice/group).

FIG. 10B shows that α-EGFR/α-CD16/α-4-1BB EPI NPs effectively inhibit the growth of A431 xenograft tumors in T cell-deficient Nu mice. FIG. 10B left panel: average tumor curves of A431 tumor-bearing mice after treatment with drug-free α-EGFR/α-CD16/α-4-1BB NPs, α-EGFR/α-CD16/α-4-1BB EPI NPs, and α-EGFR/α-CD16/α-4-1BB NPs plus free EPI. Statistical significances were calculated via one-way ANOVA with a Tukey post-hoc test. *p<0.05. FIG. 10B right panel: survival curves of A431 tumor-bearing mice after treatment with drug-free α-EGFR/α-CD16/α-4-1BB NPs, α-EGFR/α-CD16/α-4-1BB EPI NPs, and α-EGFR/α-CD16/α-4-1BB NPs plus free EPI. n=6 per group. Statistical significances were calculated via the log-rank (Mantel-Cox) test. * p<0.05.

FIG. 10C shows that EGFR-targeted nanoengagers (α-EGFR/α-CD16/α-4-1BB NPs) effectively inhibit the growth of EGFR-overexpressing MB468 tumor in vivo. Lines show individual tumor growth curves recorded for MB468 bearing Nu mice after receiving different treatments (n=6 mice/group).

FIG. 10D shows that α-EGFR/α-CD16/α-4-1BB EPI NPs effectively inhibit the growth of MB468 xenograft tumor in T cell-deficient Nu mice. Average tumor curves of MB468 tumor-bearing mice after treatment with drug-free α-EGFR/α-CD16/α-4-1BB NPs, α-EGFR/α-CD16/α-4-1BB EPI NPs, and α-EGFR/α-CD16/α-4-1BB NPs plus free EPI are shown. Statistical significances were calculated via one-way ANOVA with a Tukey post-hoc test. *p<0.05.

FIG. 10E shows that EGFR-targeted nanoengagers improve chemoimmunotherapy by guiding NK cells to attack EGFR-overexpressed tumors in vivo. Lines show individual tumor growth curves of HT29 and Raji of dual-xenograft tumor model in T-cell deficient Nu mice after receiving different treatments (n=5 for the non-treatment control group, n=7 for all other treatment groups).

FIG. 10F shows the biodistribution of different antibody-functionalized Cy5-labeled NPs in A431 tumor-bearing Nu mice. FIG. 10F panel A shows ex vivo NIR fluorescence image of different concentrations of injected Cy5-labeled NPs and the plot of concentration-dependent photon efficiency of different Cy5-labeled NPs. The photon efficiency increases linearly with the concentration of injected Cy5-labeled NPs. FIG. 10F panel B shows ex vivo fluorescence images of A431 xenograft tumor and key organs preserved from non-treated mice and mice administered with α-EGFR (100 μg per mouse) plus Cy5-labeled α-CD16/α-4-1BB NPs (6 mg NPs contained 100 μg of each antibody per mouse), Cy5-labeled α-EGFR NPs (2 mg NPs contained 100 μg conjugated α-EGFR per mouse) plus Cy5-labeled α-CD16/α-4-1BB NPs (4 mg contained 100 μg of each antibody per mouse), and Cy5-labeled α-EGFR/α-CD16/α-4-1BB NPs (6 mg NPs contained 100 μg of each antibody per mouse). Preserved tissues from left to right: A431 xenograft tumor, liver, spleen, kidney, lung, and heart. Images were recorded used a light source excited at 605±20 nm. Fluorescence emission was recorded at 690±20 nm.

FIGS. 11A-11D show that EGFR-targeted nanoengagers improve chemoimmunotherapy by recruiting NK cells to the tumor and increasing dsDNA breaks. FIG. 11A shows biodistribution of Cy5-labeled and EPI-encapsulated nanoengagers in an A431 tumor model in Nu mice recorded 40 h after i.v. tail vein administration of different immunotherapeutics/chemo-immunotherapeutics (n=5 for all control and experimental groups, except n=6 for the groups administered with Cy5-labeled α-EGFR/α-CD16/α-4-1BB NPs, α-EGFR/α-CD16/α-4-1BB EPI NPs, and α-EGFR/α-CD16/α-4-1BB NPs plus free EPI, and n=4 for the group administered with α-EGFR EPI NPs). Data represent the mean±SEM. Statistical significances were calculated via two-way ANOVA with a Tukey post-hoc test. *p<0.05. FIGS. 11B-11C shows quantification of immunofluorescence images of α-NK1.1- and α-EGFR-co-stained A431 tumor sections (FIG. 11B) and α-γ-H2AX-stained (FIG. 11D) and preserved 40 h after treatment. FIG. 11D shows serum TNF-a and INF-γ levels recorded for A431 tumor-bearing Nu mice 40 h after i.v. administration of different immunotherapeutics/chemoimmunotherapeutics.

FIG. 11E shows the biodistribution of different antibody-functionalized EPI-encapsulated NPs in A431 tumor-bearing Nu mice. FIG. 11E panel A shows ex vivo NIR fluorescence image of different concentrations of injected EPI and the plot of concentration-dependent photon efficiency of free EPI. The photon efficiency increases linearly with the concentration of injected EPI. FIG. 11E panel B shows ex vivo fluorescence images of A431 xenograft tumor and key organs preserved from non-treated mice and mice administered with free EPI (160 μg of EPI per mouse), α-EGFR/α-CD16/α-4-1BB EPI NPs (160 μg of encapsulated EPI per total 6 mg NPs contained 100 μg of each antibody per mouse), α-EGFR/α-CD16/α-4-1BB NPs (6 mg NPs contained 100 μg of each antibody per mouse) plus free EPI (160 μg of EPI per mouse), α-EGFR EPI NPs (160 μg of EPI per 6 mg NPs contained 100 μg of conjugated α-EGFR per mouse), and α-EGFR EPI NPs (160 μg of EPI per 6 mg NPs contained 100 μg of conjugated α-EGFR per mouse) plus α-CD16/α-4-1BB NPs (4 mg NPs contained 100 μg of each antibody per mouse). Preserved tissues from left to right: A431 xenograft tumor, liver, spleen, kidney, lung, and heart. Images were recorded using a light source excited at 465 ±20 nm. Fluorescence emission was recorded at 590±20 nm.

### DETAILED DESCRIPTION OF THE INVENTION

The present subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 C.F.R. § 1.822 and established usage.

Except as otherwise indicated, standard methods known to those skilled in the art may be used for cloning genes, amplifying and detecting nucleic acids molecules, and the like. Such techniques are known to those skilled in the art. See, e.g., Sambrook et al., Molecular Cloning: A Laboratory Manual 2nd Ed. (Cold Spring Harbor, N.Y., 1989); Ausubel et al. Current Protocols in Molecular Biology (Green Publishing Associates, Inc. and John Wiley & Sons, Inc., New York).

All publications, patent applications, patents, accession numbers and other references mentioned herein are incorporated by reference herein in their entirety.

### Definitions

While the following terms are believed to be well understood by one of ordinary skill in the art, the following definitions are set forth to facilitate explanation of the presently disclosed subject matter.

Following long-standing patent law convention, the terms “a” and “an” and “the” can mean one or more than one when used in this application, including the claims.

Unless otherwise indicated, all numbers expressing quantities of size, biomarker concentration, probability, percentage, and so forth used in the specification and claims are to be understood as being modified in all instances by the term “about.” For example, the amounts can vary by about 10%, 5%, 1%, or 0.5%. Accordingly, unless indicated to the contrary, the numerical parameters set forth in this specification and attached claims are approximations that can vary depending upon the desired properties sought to be obtained by the presently disclosed subject matter.

The term “and/or” when used in describing two or more items or conditions refers to situations where all named items or conditions are present or applicable, or to situations wherein only one (or less than all) of the items or conditions is present or applicable. Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of the length of a polynucleotide or polypeptide sequence, dose, time, temperature, and the like, is meant to encompass variations of ±20%, ±10%, ±5%, ±1%, ±0.5%, or even ±0.1% of the specified amount.

As used herein, the term “comprising,” which is synonymous with “including,” “containing,” and “characterized by,” is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, or ingredient not specified in the claim. When the phrase “consisting of” or “consists of” appears in a clause of the body of a claim, rather than immediately following the preamble, it limits only the element set forth in that clause; other elements are not excluded from the claim as a whole. As used herein, the phrase “consisting essentially of” limits the scope of a claim to the specified materials or steps, plus those that do not materially affect the basic and novel characteristic(s) of the claimed subject matter.

With respect to the terms “comprising,” “consisting essentially of,” and “consisting of,” where one of these three terms is used herein, the presently disclosed subject matter can include the use of any of the other terms.

As used herein, the terms “subject” and “patient” are used interchangeably herein and refer to both human and nonhuman animals. A subject of this invention can be any subject that is susceptible to a disorder that can benefit by the methods and compositions of the present invention and/or be treated for a disorder by the methods and compositions of the present invention. In some embodiments, the subject of any of the methods of the present invention is a mammal. The term “mammal” as used herein includes, but is not limited to, humans, primates, non-human primates (e.g., monkeys and baboons), cattle, sheep, goats, pigs, horses, cats, dogs, rabbits, rodents (e.g., rats, mice, hamsters, and the like), etc. Human subjects include neonates, infants, juveniles, and adults. As a further option, the subject can be a laboratory animal and/or an animal model of disease. Preferably, the subject is a human. The subject may be of any gender, any ethnicity and any age.

A “subject in need thereof” or “a subject in need of” is a subject known to have, or is suspected of having or developing or is at risk of having or developing disorder that can be treated by the methods and compositions of the present invention, or would benefit from the delivery of a particle and/or composition including those described herein.

The term “administering” or “administered” as used herein is meant to include topical, parenteral and/or oral administration, all of which are described herein. Parenteral administration includes, without limitation, intravenous, subcutaneous and/or intramuscular administration (e.g., skeletal muscle or cardiac muscle administration). It will be appreciated that the actual method and order of administration will vary according to, inter alia, the particular preparation of compound(s) being utilized, and the particular formulation(s) of the one or more other compounds being utilized. The optimal method and order of administration of the compositions of the invention for a given set of conditions can be ascertained by those skilled in the art using conventional techniques and in view of the information set out herein.

The term “administering” or “administered” also refers, without limitation, to oral, sublingual, buccal, transnasal, transdermal, rectal, intramuscular, intravenous, intraarterial (intracoronary), intraventricular, intrathecal, and subcutaneous routes. In accordance with good clinical practice, the instant compounds can be administered at a dose that will produce effective beneficial effects without causing undue harmful or untoward side effects, i.e., the benefits associated with administration outweigh the detrimental effects.

Also as used herein, the terms “treat,” “treating” or “treatment” refer to any type of action that imparts a modulating effect, which, for example, can be a beneficial and/or therapeutic effect, to a subject afflicted with a condition, disorder, disease or illness, including, for example, improvement in the condition of the subject (e.g., in one or more symptoms), delay in the progression of the disorder, disease or illness, and/or change in clinical parameters of the condition, disorder, disease or illness, etc., as would be well known in the art.

Additionally as used herein, the terms “proactive,” “prevent,” “preventing” or “prevention” refer to any type of action that results in the absence, avoidance and/or delay of the onset and/or progression of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present invention.

An “effective amount” or “therapeutically effective amount” refers to an amount of a compound or composition of this invention that is sufficient to produce a desired effect, which can be a therapeutic and/or beneficial effect. In general, a “therapeutically effective amount” or “treatment effective amount” refers to an amount that is a sufficient, but non-toxic, amount of the active ingredient (i.e., particles of this invention) to achieve the desired effect, which, for example, can be a reduction or elimination in the severity and/or frequency of symptoms and/or improvement or remediation of damage, or otherwise prevent, hinder, retard or reverse the progression of a disease or any other undesirable symptom. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an effective amount or therapeutically effective amount in any individual case can be determined by one of ordinary skill in the art by reference to the pertinent texts and literature and/or by using routine experimentation. (See, for example, Remington, The Science and Practice of Pharmacy (latest edition)). The term “biologically active” as used herein means an enzyme or protein having structural, regulatory, or biochemical functions of a naturally occurring molecule.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed.

“Amino acid sequence” and terms such as “peptide,” “polypeptide,” and “protein” are used interchangeably herein, and are not meant to limit the amino acid sequence to the complete, native amino acid sequence (i.e., a sequence containing only those amino acids found in the protein as it occurs in nature) associated with the recited protein molecule. The proteins and protein fragments of the presently disclosed subject matter can be produced by recombinant approaches or can be isolated from a naturally occurring source. The protein fragments can be any size, and for example can range in size from four amino acid residues to the entire amino acid sequence minus one amino acid.

The terms “antibody” and “immunoglobulin” include antibodies or immunoglobulins of any isotype, fragments of antibodies that retain specific binding to antigen, including but not limited to Fab, Fv, single chain Fv (scFv), Fc, and Fd fragments, chimeric antibodies, humanized antibodies, single-chain antibodies, and fusion proteins including an antigen-binding portion of an antibody and a non-antibody protein. The antibodies can in some embodiments be detectably labeled, e.g., with a radioisotope, an enzyme which generates a detectable product, a fluorescent protein, and the like. The antibodies can in some embodiments be further conjugated to other moieties, such as members of specific binding pairs, e.g., biotin (member of biotin-avidin specific binding pair), and the like. Also encompassed by the terms are Fab′, Fv, F(ab′)₂, and other antibody fragments that retain specific binding to antigen (e.g., any antibody fragment that comprises at least one paratope).

Antibodies can exist in a variety of other forms including, for example, Fv, Fab, and (Fab′)₂, as well as bi-functional (i.e., bi-specific) hybrid antibodies (see e.g., Lanzavecchia et al., 1987) and in single chains (see e.g., Huston et al., 1988 and Bird et al., 1988, each of which is incorporated herein by reference in its entirety). See generally, Hood et al., 1984, and Hunkapiller & Hood, 1986. The phrase “detection molecule” is used herein in its broadest sense to include any molecule that can bind with sufficient specificity to a biomarker to allow for detection of the particular biomarker. To allow for detection can mean to determine the presence or absence of the particular biomarker member and, in some embodiments, can mean to determine the amount of the particular biomarker. Detection molecules can include antibodies, antibody fragments, and nucleic acid sequences.

As used herein, the term “target” comprises an endogenous or exogenous molecule of interest, e.g., a “marker.” A target may be a marker that is exclusively or primarily associated with one or a few tissue types, with one or a few cell types, with one or a few diseases, and/or with one or a few developmental stages. In some embodiments, a target can comprise a protein (e.g., a cell surface receptor, transmembrane protein, glycoprotein, etc.), a carbohydrate (e.g., a glycan moiety, glycocalyx, etc.), a lipid (e.g., steroid, phospholipid, etc.), and/or a nucleic acid (e.g. DNA, RNA, etc.). In some embodiments, a target may be an NK cell target (e.g., a “first target”). In some embodiments, a target (i.e., marker) may be a molecule that is present exclusively or in higher amounts on a malignant cell, e.g., a tumor antigen (e.g., a “second target”). In some embodiments, a target may be a prostate cancer marker. In certain embodiments, the prostate cancer marker is prostate specific membrane antigen (PSMA), a 100 kDa transmembrane glycoprotein that is expressed in most prostatic tissues, but is more highly expressed in prostatic cancer tissue than in normal tissue. In some embodiments, a target may be a breast cancer marker. In some embodiments, a target may be a colon cancer marker. In some embodiments, a target may be a rectal cancer marker. In some embodiments, a target may be a lung cancer marker. In some embodiments, a target may be a pancreatic cancer marker. In some embodiments, a target may be an ovarian cancer marker. In some embodiments, a target may be a bone cancer marker. In some embodiments, a target may be a renal cancer marker. In some embodiments, a target may be a liver cancer marker. In some embodiments, a target may be a neurological cancer marker. In some embodiments, a target may be a gastric cancer marker. In some embodiments, a target may be a testicular cancer marker. In some embodiments, a target may be a head and neck cancer marker. In some embodiments, a target may be an esophageal cancer marker. In some embodiments, a target may be a cervical cancer marker.

As used herein, the terms “nanoparticle” and/or “nanosphere” describe a polymeric particle or sphere in the nanometer size range. The term “microparticle” or “microsphere” as used herein describes a particle or sphere in the micrometer size range. Both types of particles or spheres can be used as drug carriers into which drugs, imaging agents and/or antigens may be incorporated in the form of solid solutions or solid dispersions or onto which these materials may be absorbed, encapsulated, and/or chemically bound.

The term “targeting agent” as used herein comprises an agent which binds to a specific marker (i.e., target). A targeting agent (e.g., targeting moiety) of the present invention may be a nucleic acid (e.g., aptamer), polypeptide (e.g., antibody), glycoprotein, small molecule, carbohydrate, lipid, etc. For example, a targeting agent can be an aptamer, which is generally an oligonucleotide (e.g., DNA, RNA, or an analog or derivative thereof) that binds to a particular target, such as a polypeptide. In some embodiments, the targeting agent may be a peptide or a polypeptide (e.g., an antibody or portion of an antibody that specifically recognizes a tumor marker (e.g., a target on a cancer cell surface)). In some embodiments, the targeting agent may be an antibody or a fragment thereof. In some embodiments, the targeting agent may be an Fc fragment of an antibody.

### Particles and Compositions

The current disclosure describes the utilization of nanoparticles that comprise NK cell-binding targets and cancer cell-binding targets in a single particle, and relates to the approach of combining NK cell immune response activation and direct treatment of cancer cells, optionally with therapeutic agents.

Thus, in one embodiment, the present invention provides a particle (e.g., a nanoparticle) comprising: at least one first targeting agent that binds a first target on an NK cell surface (e.g., wherein the first targeting agent binds its respective first target); and at least one second targeting agent that binds a second target on a cancer cell surface(e.g., wherein the second targeting agent binds its respective second target), wherein the second targeting agent is different from the first targeting agent (e.g., wherein the respective first target and the respective second target are different targets). In some embodiments, the particle of the present invention can comprise more than one first target agents, e.g., at least two first targeting agents (including 2, 3, 4, 5, 6, 7, 8, 9, 10, 11, 12, 13, 14, 15, etc.), wherein the at least two first targeting agents bind different first targets on an NK cell surface, e.g., wherein the at least two first targeting agents are two different first targeting agents. In some embodiments, a first target agent may comprise a multiplicity of the same first binding agents, e.g., about 5, 10, 50, 100, 500, 1000, 2000, 5000, 10,000, or more of the same first binding agents or any value or range therein.

Types of particles of this invention include, but are not limited to, polymer nanoparticles such as PLGA-based, PLA-based, polysaccharide-based (dextran, cyclodextrin, chitosan, heparin), dendrimer, hydrogel; lipid-based nanoparticles such as lipid nanoparticles, lipid hybrid nanoparticles, liposomes, micelles; inorganics-based nanoparticles such as superparamagnetic iron oxide nanoparticles, metal nanoparticles, platin nanoparticles, calcium phosphate nanoparticles, quantum dots; carbon-based nanoparticles such as fullerenes, carbon nanotubes; and protein-based complexes with nanoscales. Types of microparticles of this invention include but are not limited to particles with sizes at micrometer scale that are polymer microparticles including but not limited to, PLGA-based, PLA-based, polysaccharide-based (dextran, cyclodextrin, chitosan, heparin), dendrimer, hydrogel; lipid-based microparticles such as lipid microparticles, micelles; inorganics-based microparticles such as superparamagnetic iron oxide microparticles, platin microparticles and the like as are known in the art. These particles may be generated and/or have materials be absorbed, encapsulated, or chemically bound through known mechanisms in the art, such as those described in Au et al. 2019 ACS Cent. Sci. 5(1):122-144 and Au et al. 2018 ACS Nano 12(2):1544-1563, the disclosures of which are incorporated herein by reference in their entirety.

The particle of this invention may be any sized particle comprising at least one first targeting agent that binds a first target on an NK cell surface; and at least one second targeting agent that binds a second target on a cancer cell surface, e.g., a microparticle, e.g., a nanoparticle. In some embodiments, the particle can be a nanoparticle, e.g., wherein the diameter of the particle (e.g., nanoparticle) is 1 μm or less, e.g., wherein the diameter of the nanoparticle is 1 nm, 5 nm, 10 nm, 20 nm, 30 nm, 40 nm, 50 nm, 60 nm, 70 nm, 80 nm, 90 nm, 100 nm, 125 nm, 150 nm, 175 nm, 200 nm, 225 nm, 250 nm, 275 nm, 300 nm, 325 nm, 350 nm, 375 nm, 400 nm, 425 nm, 450 nm, 475 nm, 500 nm, 525 nm, 550 nm, 575 nm, 600 nm, 625 nm, 650 nm, 675 nm, 700 nm, 725 nm, 725 nm, 750 nm, 775 nm, 800 nm, 825 nm, 850 nm, 875 nm, 900 nm, 925 nm, 950 nm, 960 nm, 970 nm, 980 nm, 990 nm, 995 nm, 996 nm, 997 nm, 998 nm, or 999 nm, or any value or range therein. In some embodiments, the diameter of the nanoparticle can be about 5 nm to about 750 nm, about 10 nm to about 500 nm, about 5 nm to about 999 nm, or about 50 nm to about 900 nm. In some embodiments, the diameter of the particle can be the average diameter of a population of particles, e.g., wherein the average diameter of the nanoparticle can be e.g., about 5 nm to about 750 nm, about 10 nm to about 500 nm, about 5 nm to about 999 nm, or about 50 nm to about 900 nm, or any value or range therein. A nanoparticle or nanosphere of this invention can have a diameter of 100 nm or less (e.g., in a range from about 1 nm to about 100 nm). In some embodiments, a particle with dimensions more than 100 nm can still be called a nanoparticle. Thus, an upper range for nanoparticles can be about 1μm, and in some embodiments, 500 nm. A microparticle or microsphere of this invention can have a diameter of about 0.5 micrometers to about 100 micrometers.

A first targeting agent of the present invention can be any targeting agent that binds a target on an NK cell surface. A target on an NK cell surface may be referred to herein as a “first target,” wherein each first targeting agent binds a respective first target (e.g., a binding partner of the first targeting agent). In some embodiments, the first target on the NK cell surface can be CD16, 4-1BB, NKG2D, TRAIL, NKG2C, CD137, OX40, CD27, KIRs, NKG2a, dnam-1, 2b4, NKp30a, NKp30b, NKp30c, an antibody Fc component, or any combination thereof, as well as any other marker on an NK cell surface that is now known or later identified. In some embodiments, the target on the NK cell surface can be CD16 and/or 4-1BB. For example, in some embodiments, a particle of the present invention may comprise a first targeting agent that binds CD16 (e.g., the respective first target of the first targeting agent) and further comprise another different first targeting agent that binds 4-1BB (e.g., the respective first target of the different first targeting agent). Thus, in embodiments of this invention, a nanoparticle of this invention can comprise multiple first targeting agents (e.g., 2, 3, 4, 5, 6, 7, 8, 9, 10, etc.) that are different from one another.

As used herein, a “respective first target” is the specific first target that will be recognized by a particular first targeting agent. For example a first targeting agent having specificity for first target X will recognize and bind first target X, therefore first target X is the respective first target of the first targeting agent.

A second targeting agent of the present invention can be any targeting agent that binds a target on a cancer cell surface. A target on a cancer cell surface may be referred to herein as a “second target,” wherein each second targeting agent binds a respective second target (e.g., a binding partner of the second targeting agent). Non-limiting exemplary cancer and tumor cell antigens are described in S.A. Rosenberg (Immunity 10:281 (1991)). Other illustrative cancer and tumor antigens include, but are not limited to: BRCA1 gene product, BRCA2 gene product, gp100, tyrosinase, GAGE-1/2, BAGE, RAGE, LAGE, NY-ESO-1, CDK-4, β-catenin, MUM-1, Caspase-8, KIAA0205, HPVE, SART-1, PRAME, p15, melanoma tumor antigens (Kawakami et al., (1994) Proc. Natl. Acad. Sci. USA 91:3515; Kawakami et al., (1994) J. Exp. Med., 180:347; Kawakami et al., (1994) Cancer Res. 54:3124), MART-1, gp100 MAGE-1, MAGE-2, MAGE-3, CEA, TRP-1, TRP-2, P-15, tyrosinase (Brichard et al., (1993) J. Exp. Med. 178:489); HER-2/neu gene product (U.S. Pat. No. 4,968,603), CA 125, LK26, FB5 (endosialin), TAG 72, AFP, CA19-9, NSE, DU-PAN-2, CA50, SPan-1, CA72-4, HCG, STN (sialyl Tn antigen), c-erbB-2 proteins, PSA, L-CanAg, estrogen receptor, milk fat globulin, p53 tumor suppressor protein (Levine, (1993) Ann. Rev. Biochem. 62:623); mucin antigens (International Patent Publication No. WO 90/05142); telomerases; nuclear matrix proteins; prostatic acid phosphatase; papilloma virus antigens; and/or antigens now known or later discovered to be associated with the cancers including but not limited to the following cancers: adenocarcinoma, thymoma, sarcoma, brain cancer (e.g., glioblastoma), head and neck cancer, esophageal cancer, gastric cancer, lung cancer (e.g., small cell lung cancer, non-small cell lung cancer), bladder cancer, kidney cancer (e.g., renal cell carcinoma), liver cancer (e.g., hepatocellular carcinoma), pancreatic cancer, uterine cancer, ovarian cancer, cervical cancer, anal cancer, melanoma, prostate cancer, breast cancer, blood cell cancer (e.g., leukemia, lymphoma (e.g., non-Hodgkin's lymphoma, Hodgkin's lymphoma), multiple myeloma), colorectal cancer, and any other cancer or malignant condition now known or later identified (see, e.g., Rosenberg, (1996) Ann. Rev. Med. 47:481-91). In some embodiments, the target on the cancer cell surface can be, but is not limited to, EGFR, PSMA, Nectin-4, mucin, HER-2, CD30, CD22, or any combination thereof. Thus, in embodiments of this invention, a nanoparticle of this invention can comprise multiple second targeting agents (e.g., 2, 3, 4, 5, 6, 7, 8, 9, 10, etc.) that are different from one another.

As used herein, a “respective second target” is the specific second target that will be recognized by a particular second targeting agent. For example a second targeting agent having specificity for second target X will recognize and bind second target X, therefore second target X is the respective second target of the second targeting agent.

The cancer cell of this invention can be a cell from any cancer. In some embodiments, the cancer cell can be a cell from an adenocarcinoma, thymoma, sarcoma, brain cancer (e.g., glioblastoma), head and neck cancer, thyroid cancer, sarcoma, squamous cell carcinoma, skin cancer, salivary gland cancer, esophageal cancer, gastric cancer, lung cancer (e.g., small cell lung cancer, non-small cell lung cancer), bladder cancer, kidney cancer (e.g., renal cell carcinoma), liver cancer (e.g., hepatocellular carcinoma), pancreatic cancer, uterine cancer, ovarian cancer, cervical cancer, anal cancer, melanoma, prostate cancer, testicular cancer, breast cancer, blood cell cancer (e.g., leukemia, lymphoma (e.g., non-Hodgkin's lymphoma, Hodgkin's lymphoma), multiple myeloma), colorectal cancer, and any other cancer or malignant condition now known or later identified.

In some embodiments, a particle of the present invention may comprise further targeting agents, e.g., a third targeting agent, a fourth targeting agent, a fifth targeting agent, etc., which bind additional targets not expressed on an NK cell surface and/or on a cancer cell surface (e.g., a respective third target, a respective fourth target, a respective fifth target, etc.).

In some embodiments of the present invention, the targeting agent may be an antibody or active fragment thereof. In some embodiments, the first targeting agent and/or the second targeting agent may be an antibody or active fragment thereof. In some embodiments, each of the targeting agents is an antibody or active fragment thereof. In some embodiments, the antibody or active fragment thereof is selected from the group consisting of a monoclonal antibody, a Fab fragment, a Fab'-SH fragment, a FV fragment, a scFV fragment, a (Fab′)₂ fragment, an Fc-fusion protein, and any combination thereof.

In some embodiments, a particle (e.g., nanoparticle) of the present invention comprises an antibody or active fragment thereof that specifically binds to CD16, an antibody or active fragment thereof that specifically binds to 4-1BB, and/or an antibody or active fragment thereof that specifically binds to EGFR.

In some embodiments, a particle (e.g., nanoparticle) of the present invention may further comprise a therapeutic agent. Non-limiting exemplary therapeutic agents include small molecules (e.g., cytotoxic agents), nucleic acids (e.g., RNAi agents), proteins (e.g., antibodies), peptides, lipids, carbohydrates, hormones, metals, radioactive elements and compounds, drugs, vaccines, immunological agents, etc., and/or combinations thereof. In some embodiments, the therapeutic agent may be an agent useful in the treatment of cancer, e.g., a chemotherapeutic. Nonlimiting examples of chemotherapeutic agents include daunomycin, cisplatin, oxaliplatin, carboplatin, verapamil, cytosine arabinoside, aminopterin, democolcine, tamoxifen, actinomycin D, alkylating agents (including, without limitation, nitrogen mustards, ethylenimine derivatives, alkyl sulfonates, nitrosoureas and triazenes): Uracil mustard, Chlormethine, Cyclophosphamide (Cytoxan®), Ifosfamide, Melphalan, Chlorambucil, Pipobroman, Triethylene-melamine, Triethylenethiophosphoramine, Busulfan, Carmustine, Lomustine, Streptozocin, Dacarbazine, and Temozolomide; Antimetabolites (including, without limitation, folic acid antagonists, pyrimidine analogs, purine analogs and adenosine deaminase inhibitors): Methotrexate, 5-fluorouracil (5-FU), Floxuridine, Cytarabine, 6-Mercaptopurine, 6-Thioguanine, Fludarabine phosphate, Pentostatine, and Gemcitabine, Natural products and their derivatives (for example, vinca alkaloids, antitumor antibiotics, enzymes, lymphokines and epipodophyllotoxins): Vinblastine, Vincristine, Vindesine, Bleomycin, Dactinomycin, Daunorubicin, Doxorubicin, Epirubicin, Idarubicin, Ara-C, paclitaxel (paclitaxel is commercially available as Taxol®), docetaxel, Mithramycin, Deoxyco-formycin, Mitomycin-C, L-Asparaginase, Interferons (especially IFN-a), Etoposide, and Teniposide; Other anti-proliferative cytotoxic agents are navelbene, CPT-11, anastrazole, letrazole, capecitabine, reloxafine, cyclophosphamide, ifosamide, and droloxafine. Additional anti-proliferative cytotoxic agents include, but are not limited to, melphalan, hexamethyl melamine, thiotepa, cytarabine, idatrexate, trimetrexate, dacarbazine, L-asparaginase, camptothecin, topotecan, bicalutamide, flutamide, leuprolide, pyridobenzoindole derivatives, interferons, interleukins, antiproliferative cytotoxic agents (including, but not limited to, EGFR inhibitors, Her-2 inhibitors, CDK inhibitors, and trastuzumab). In some embodiments, the therapeutic agent is a chemotherapeutic agent selected from the group consisting of epirubicin (EPI), doxorubicin, cisplatin, oxaliplatin, carboplatin, daunorubicin, taxol, docetaxel, gemcitabine, 5-fluorouracil, mitomycin, cytarabine, cytoxan, and any combination thereof.

In some embodiments, the present invention provides a composition comprising a particle (e.g., nanoparticle) of the present invention and a pharmaceutically acceptable carrier. “Pharmaceutically acceptable,” as used herein, means a material that is not biologically or otherwise undesirable, i.e., the material may be administered to a subject along with the compositions of this invention, without causing substantial deleterious biological effects or interacting in a deleterious manner with any of the other components of the composition in which it is contained. The material would naturally be selected to minimize any degradation of the active ingredient and to minimize any adverse side effects in the subject, as would be well known to one of skill in the art (see, e.g., Remington's Pharmaceutical Science; latest edition). Exemplary pharmaceutically acceptable carriers for the compositions of this invention include, but are not limited to, sterile pyrogen-free water and sterile pyrogen-free physiological saline solution, as well as other carriers suitable for injection into and/or delivery to a subject of this invention, particularly a human subject, as would be well known in the art.

The present invention also provides methods for delivering a particle (e.g., nanoparticle) of the present invention to a cell or a subject for therapeutic or research purposes in vitro, ex vivo, and/or in vivo.

Thus, in some embodiments, the present invention provides a method of activating an NK cell, comprising contacting the NK cell with a particle (e.g., nanoparticle) or composition of the present invention under conditions whereby the first targeting agent binds the first target (e.g., the respective target of the first targeting agent) on the NK cell surface.

In some embodiments, the present invention provides a method of inducing an NK cell immune response, comprising contacting the NK cell with a particle (e.g., nanoparticle) or composition of the present invention under conditions whereby the first targeting agent binds the first target (e.g., the respective target of the first targeting agent) on the NK cell surface.

In some embodiments, the present invention provides a method of inducing cytotoxicity in a cancer cell, comprising contacting the cancer cell with a particle (e.g., nanoparticle) or composition of the present invention under conditions whereby the second targeting agent binds the second target (e.g., the respective target of the second targeting agent) on the surface of the cancer cell. In some embodiments, the nanoparticle or composition may further comprise a therapeutic agent, wherein contacting the cancer cell with a particle and/or composition thereby delivers the therapeutic agent to the cancer cell.

In some embodiments, the present invention provides a method of delivering a therapeutic agent to a cancer cell, comprising contacting the cancer cell with a particle (e.g., nanoparticle) or composition of the present invention comprising a therapeutic agent under conditions whereby the second targeting agent binds the second target (e.g., the respective target of the second targeting agent) on the surface of the cancer cell, thereby delivering the therapeutic to the cancer cell.

In some embodiments, the present invention provides a method of inducing an NK cell immune response in a subject in need thereof, comprising administering to the subject an effective amount of a particle (e.g., nanoparticle) or composition of the present invention under conditions whereby the first targeting agent binds the first target (e.g., the respective target of the first targeting agent) on the surface of the NK cell. In some embodiments, the particle and/or composition comprises at least two first targeting agents. In some embodiments, the NK cell that the at least two first targeting agents bind is the same NK cell.

In some embodiments, the present invention provides a method of activating NK cells in a subject in need thereof, comprising administering to the subject an effective amount of a particle (e.g., nanoparticle) or composition of the present invention under conditions whereby the first targeting agent binds the first target (e.g., the respective target of the first targeting agent) on the surface of the NK cell. In some embodiments, the particle and/or composition comprises at least two first targeting agents. In some embodiments, the NK cell that the at least two first targeting agents bind is the same NK cell.

In some embodiments of the present invention, the subject of any of the methods of the present invention has been diagnosed with cancer. In some embodiments, the cancer is selected from the group consisting of adenocarcinoma, thymoma, sarcoma, brain cancer (e.g., glioblastoma), head and neck cancer, esophageal cancer, gastric cancer, lung cancer (e.g., small cell lung cancer, non-small cell lung cancer), bladder cancer, kidney cancer (e.g., renal cell carcinoma), liver cancer (e.g., hepatocellular carcinoma), pancreatic cancer, uterine cancer, ovarian cancer, cervical cancer, anal cancer, melanoma, prostate cancer, breast cancer, blood cell cancer (e.g., leukemia, lymphoma (e.g., non-Hodgkin's lymphoma, Hodgkin's lymphoma), multiple myeloma), colorectal cancer, and any combination thereof.

In some embodiments, the present invention provides a method of treating cancer in a subject (e.g., a subject in need thereof), comprising administering to the subject an effective amount of a particle (e.g., nanoparticle) or composition of the present invention under conditions whereby the second targeting agent binds the second target (e.g., the respective target of the second targeting agent) on the surface of the cancer cell.

In some embodiments, the present invention provides a method of treating cancer in a subject in need thereof, comprising administering to the subject an effective amount of a particle (e.g., nanoparticle) or composition of the present invention under conditions whereby the first targeting agent binds the first target (e.g., the respective target of the first targeting agent) on the surface of the NK cell and whereby the second agent binds the second target (e.g., the respective target of the second targeting agent) on the surface of the cancer cell.

In some embodiments, the present invention provides a method of delivering a therapeutic agent to a cancer cell of a cancer in a subject in need thereof, comprising administering to the subject an effective amount of a particle (e.g., nanoparticle) or composition of the present invention comprising a therapeutic agent under conditions whereby the second targeting agent binds the second target (e.g., the respective target of the second targeting agent) on the surface of the cancer cell, thereby delivering the therapeutic to the cancer cell in the subject.

In some embodiments of the present invention, the particle (e.g., nanoparticle) or composition of the present invention may be administered via a route selected from the group consisting of intravenous, intramuscular, subcutaneous, topical, oral, transdermal, intraperitoneal, intrathecal, intraventricular, intraocular, intravitreal, intraorbital, intranasal, by implantation, by inhalation, by intratumoral, and any combination thereof. In some embodiments, the methods of the present invention may further comprise the step of administering to the subject an effective amount of a therapeutic agent (e.g., chemotherapeutic agent) and/or radiation therapy. Non-limiting exemplary therapeutic agents that may be administered in conjunction with administering a particle and/or composition of the present invention include small molecules (e.g. cytotoxic agents), nucleic acids (e.g. RNAi agents), proteins/peptides (e.g. antibodies), lipids, carbohydrates, hormones, metals, radioactive elements and compounds, drugs, vaccines, immunological agents, etc., and/or combinations thereof. In some embodiments, the therapeutic agent can be an agent useful in the treatment of cancer, e.g., a chemotherapeutic. Nonlimiting examples of chemotherapeutic agents include daunomycin, cisplatin, oxaliplatin, carboplatin, verapamil, cytosine arabinoside, aminopterin, democolcine, tamoxifen, Actinomycin D, Alkylating agents (including, without limitation, nitrogen mustards, ethylenimine derivatives, alkyl sulfonates, nitrosoureas and triazenes): Uracil mustard, Chlormethine, Cyclophosphamide (Cytoxan®), Ifosfamide, Melphalan, Chlorambucil, Pipobroman, Triethylene-melamine, Triethylenethiophosphoramine, Busulfan, Carmustine, Lomustine, Streptozocin, Dacarbazine, and Temozolomide; Antimetabolites (including, without limitation, folic acid antagonists, pyrimidine analogs, purine analogs and adenosine deaminase inhibitors): Methotrexate, 5-fluorouracil (5-FU), Floxuridine, Cytarabine, 6-Mercaptopurine, 6-Thioguanine, Fludarabine phosphate, Pentostatine, and Gemcitabine, Natural products and their derivatives (for example, vinca alkaloids, antitumor antibiotics, enzymes, lymphokines and epipodophyllotoxins): Vinblastine, Vincristine, Vindesine, Bleomycin, Dactinomycin, Daunorubicin, Doxorubicin, Epirubicin, Idarubicin, Ara-C, paclitaxel (paclitaxel is commercially available as Taxol®), docetaxel, Mithramycin, Deoxyco-formycin, Mitomycin-C, L-Asparaginase, Interferons (especially IFN-a), Etoposide, and Teniposide; Other anti-proliferative cytotoxic agents are navelbene, CPT-11, anastrazole, letrazole, capecitabine, reloxafine, cyclophosphamide, ifosamide, and droloxafine. Additional anti-proliferative cytotoxic agents include, but are not limited to, melphalan, hexamethyl melamine, thiotepa, cytarabine, idatrexate, trimetrexate, dacarbazine, L-asparaginase, camptothecin, topotecan, bicalutamide, flutamide, leuprolide, pyridobenzoindole derivatives, interferons, interleukins, antiproliferative cytotoxic agents (including, but not limited to, EGFR inhibitors, Her-2 inhibitors, CDK inhibitors, and trastuzumab). In some embodiments, the therapeutic agent that may be administered in conjunction with administering a particle and/or composition of the present invention is a chemotherapeutic agent selected from the group consisting of epirubicin (EPI), doxorubicin, cisplatin, oxaliplatin, carboplatin, daunorubicin, taxol, docetaxel, gemcitabine, 5-fluorouracil, mitomycin, cytarabine, cytoxan, and any combination thereof.

Additional aspects of the present invention include use of a particle (e.g., a nanoparticle) and/or composition of the present invention in activating NK cells, in inducing cytotoxicity in a cancer cell, in delivering a therapeutic agent to a cancer cell, and/or in treating cancer. Further provided herein are preparation of a medicament for use comprising a particle (e.g., a nanoparticle) and/or the composition of the present invention.

In some embodiments, further provided herein is a kit comprising a particle (e.g., a nanoparticle) and/or composition of the present invention and instructions for use.

### Pharmaceutical Compositions and Methods of Use

In some embodiments, the invention also provides compositions comprising the particles of this invention together with one or more of the following: a pharmaceutically acceptable diluent; a carrier; a solubilizer; an emulsifier; a preservative; and/or an adjuvant. Such compositions may contain an effective amount of the particles. Thus, the use of the particles as provided herein in the preparation of a pharmaceutical composition or medicament is also included. Such compositions can be used in the treatment of a variety of diseases and disorders as described herein.

Acceptable formulation components for pharmaceutical preparations are nontoxic to recipients at the dosages and concentrations employed. In addition to the particles provided herein, compositions according to the invention may contain components for modifying, maintaining or preserving, for example, the pH, osmolarity, viscosity, clarity, color, isotonicity, odor, sterility, stability, rate of dissolution or release, adsorption or penetration of the composition. Suitable materials for formulating pharmaceutical compositions include, but are not limited to, amino acids (such as glycine, glutamine, asparagine, arginine or lysine); antimicrobials; antioxidants (such as ascorbic acid, sodium sulfite or sodium hydrogen-sulfite); buffers (such as acetate, borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants.

The primary vehicle or carrier in a pharmaceutical composition may be either aqueous or non-aqueous in nature. Suitable vehicles or carriers for such compositions include water (e.g., sterile water) for injection, physiological saline solution or artificial cerebrospinal fluid, possibly supplemented with other materials common in compositions for parenteral administration. Neutral buffered saline or saline mixed with serum albumin are further exemplary vehicles.

Compositions comprising particles of this invention may be prepared for storage by mixing the selected composition having the desired degree of purity with optional formulation agents in the form of a lyophilized cake or an aqueous solution. Further, the particles may be formulated as a lyophilizate using appropriate excipients such as sucrose.

Formulation components are present in concentrations that are acceptable to the site of administration. Buffers are advantageously used to maintain the composition at physiological pH or at a slightly lower pH, typically within a pH range of from about 4.0 to about 8.5, or alternatively, between about 5.0 to 8.0. Pharmaceutical compositions can comprise TRIS buffer of about pH 6.5-8.5, or acetate buffer of about pH 4.0-5.5, which may further include sorbitol or a suitable substitute therefor.

A pharmaceutical composition may involve an effective quantity of particles of this invention in a mixture with non-toxic excipients that are suitable for the manufacture of tablets. By dissolving the tablets in sterile water, or another appropriate vehicle, solutions may be prepared in unit-dose form. Suitable excipients include, but are not limited to, inert materials, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions are in the form of sustained- or controlled-delivery formulations. Techniques for formulating a variety of other sustained- or controlled-delivery means, such as liposome carriers, bio-erodible microparticles or porous beads and depot injections can be. Sustained-release preparations may include semipermeable polymer matrices in the form of shaped articles, e.g., films, or microcapsules, polyesters, hydrogels, polylactides, copolymers of L-glutamic acid and gamma ethyl-L-glutamate, poly (2-hydroxyethyl-methacrylate), ethylene vinyl acetate or poly-D(−)-3-hydroxybutyric acid. Sustained release compositions may also include liposomes, which can be prepared by any of several methods known in the art.

The pharmaceutical composition to be used for in vivo administration typically is sterile. Sterilization may be accomplished by filtration through sterile filtration membranes. If the composition is lyophilized, sterilization may be conducted either prior to or following lyophilization and reconstitution. The composition for parenteral administration may be stored in lyophilized form or in a solution. In certain embodiments, parenteral compositions are placed into a container having a sterile access port, for example, an intravenous solution bag or vial having a stopper pierceable by a hypodermic injection needle, or a sterile pre-filled syringe ready to use for injection.

The composition may be formulated for transdermal delivery, optionally with the inclusion of microneedles, microprojectiles, patches, electrodes, adhesives, backings, and/or packaging, or formulations for jet delivery, in accordance with known techniques. See, e.g., U.S. Pat. Nos. 8,043,250; 8,041,421; 8,036,738; 8,025,898; 8,017,146.

Once the pharmaceutical composition of the invention has been formulated, it may be stored in sterile vials as a solution, suspension, gel, emulsion, solid, or as a dehydrated or lyophilized powder. Such formulations may be stored either in a ready-to-use form or in a form (e.g., lyophilized) that is reconstituted prior to administration.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The present invention provides kits for producing multi-dose or single-dose administration units. For example, kits according to the invention may each contain both a first container having a dried composition and a second container having an aqueous diluent, including for example single and multi-chambered pre-filled syringes (e.g., liquid syringes, lyosyringes or needle-free syringes).

The pharmaceutical compositions of the invention can be delivered parenterally, typically by injection. Injections can be intraocular, intraperitoneal, intraportal, intramuscular, intravenous, intrathecal, intracerebral (intra-parenchymal), intracerebroventricular, intravitreal, intraarterial, intralesional, perilesional or subcutaneous. Eye drops can be used for intraocular administration. In some instances, injections may be localized to the vicinity of a particular bone or bones to which the treatment is targeted. For parenteral administration, the chimeric protein may be administered in a pyrogen-free, parenterally acceptable aqueous solution comprising the chimeric protein in a pharmaceutically acceptable vehicle. A particularly suitable vehicle for parenteral injection is sterile distilled water in which the chimeric proteins are formulated as a sterile, isotonic solution, properly preserved.

Pharmaceutical compositions comprising the particles of this invention may be administered by bolus injection and/or continuously by infusion, by implantation device, sustained release systems or other means for accomplishing prolonged release. The pharmaceutical composition also can be administered locally via implantation of a membrane, sponge or another appropriate material onto which the desired molecule has been absorbed or encapsulated. Where an implantation device is used, the device may be implanted into any suitable tissue or organ, and delivery of the desired molecule may be via diffusion, timed-release bolus, or continuous release. The preparation may be formulated with agent, such as injectable microspheres, bio-erodible particles, polymeric compounds (such as polylactic acid; polyglycolic acid; or copoly (lactic/glycolic) acid (PLGA), beads or liposomes, that can provide controlled or sustained release of the product which may then be delivered via a depot injection. Formulation with hyaluronic acid has the effect of promoting sustained duration in the circulation.

The subject compositions comprising particles of this invention may be formulated for inhalation. In these embodiments, the particles can be formulated as a dry powder for inhalation, or particle inhalation solutions may also be formulated with a propellant for aerosol delivery, such as by nebulization.

Certain pharmaceutical compositions of the invention can be delivered through the digestive tract, such as orally. The particles of this invention that are administered in this fashion may be formulated with or without those carriers customarily used in the compounding of solid dosage forms such as tablets and capsules. A capsule may be designed to release the active portion of the formulation at the point in the gastrointestinal tract when bioavailability is maximized and pre-systemic degradation is minimized. Additional agents can be included to facilitate absorption of the particles. For oral administration, modified amino acids may be used to confer resistance to digestive enzymes. Diluents, flavorings, low melting point waxes, vegetable oils, lubricants, suspending agents, tablet disintegrating agents, and binders may also be employed.

The subject compositions comprising particles also may be used ex vivo. In such instances, cells, tissues or organs that have been removed from the subject are exposed to or cultured with the particles. The cultured cells may then be implanted back into the subject or a different subject or used for other purposes.

In some embodiments, in order to decrease the chance of an immunological response, the particles of this invention may be encapsulated to avoid infiltration of surrounding tissues. Encapsulation materials are typically biocompatible, semi-permeable polymeric enclosures or membranes that allow the release of the particles but prevent the destruction of the cells by the patient's immune system or by other detrimental factors from the surrounding tissues.

The pharmaceutical compositions that are provided can be administered for prophylactic and/or therapeutic treatments. In general, toxicity and therapeutic efficacy of the particles of this invention can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred.

The data obtained from cell culture and/or animal studies can be used in formulating a range of dosages for subjects for treatment. The dosage of the active ingredient typically falls within a range of circulating concentrations that include the ED₅₀ with little or no toxicity. The dosage can vary within this range depending upon the dosage form employed and the route of administration utilized.

The effective amount of a pharmaceutical composition comprising particles of this invention to be employed therapeutically or prophylactically will depend, for example, upon the therapeutic context and objectives. One skilled in the art will appreciate that the appropriate dosage levels for treatment, according to certain embodiments, will thus vary depending, in part, upon the composition being delivered, the indication for which the particles are being used, the route of administration, and the size (body weight, body surface or organ size) and/or condition (the age and general health) of the subject. A clinician may titer the dosage and modify the route of administration to obtain the optimal therapeutic effect. Typical dosages for administration of the particles of this invention range from about 0.001 mg/kg to 2000 mg/kg. For example, in some embodiments, the particles can be administrated intravenously every one to three weeks.

The dosing frequency will depend upon the pharmacokinetic parameters of particles in the formulation. For example, a clinician will administer the composition until a dosage is reached that achieves the desired effect. The composition may therefore be administered as a single dose or as two or more doses (which may or may not contain the same amount of the desired molecule) over time, or as a continuous infusion via an implantation device or catheter. Treatment may be continuous over time or intermittent. Further refinement of the appropriate dosage is routinely made by those of ordinary skill in the art and is within the ambit of tasks routinely performed by them. Appropriate dosages may be ascertained through use of appropriate dose-response data.

In some embodiments, the particles can be administered in combination with one or more other therapeutic agents and/or different therapies. Examples of therapeutic agents include, but are not limited to, an anti-infectious agent (e.g., an anti-septic agent, anti-biotic agent, and/or anti-fungal agent), an anti-inflammatory agent, and/or an immunomodulatory agent. The therapeutic agent can be administered simultaneously with the particles and/or can be administered at a different time point. The route of administration of the therapeutic agent can be the same or different as the route of administration of the particles.

To treat a disorder of this invention, a composition comprising the particles of this invention may be administered to the subject in need thereof in an amount and for a time sufficient to induce a sustained improvement in at least one indicator that reflects the severity of the disorder. For example, the particles can be administered about every 1, 2, 3, 4, 5, 6, 7, 8, 9, or 10 or more days and/or weeks. In other embodiments, the particles can be about 1, 2, 3, 4, 5, 6, 7, 8, 9 or 10 or more times a week and/or month and/or year. In some embodiments, an improvement is considered “sustained” if the subject exhibits the improvement on at least two occasions separated by at least one to seven days, or in some instances one to six weeks. The appropriate interval will depend to some extent on what disease condition is being treated. It is within the purview of those skilled in the art to determine the appropriate interval for determining whether the improvement is sustained.

Kits that include particles of this invention and/or a pharmaceutical composition as described herein are also provided herein. Some kits include particles and/or compositions in a container (e.g., vial or ampule), and may also include instructions for use of the particles and/or composition in the various methods disclosed above. The particles and/or composition can be in various forms, including, for instance, as part of a solution or as a solid (e.g., lyophilized powder). The instructions may include a description of how to prepare (e.g., dissolve or resuspend) the particles in an appropriate fluid and/or how to administer the particles for the treatment of the diseases and disorders described herein.

The kits may also include various other components, such as buffers, salts, complexing metal ions and other agents described above in the section on pharmaceutical compositions. These components may be included with the chimeric protein or may be in separate containers. The kits may also include other therapeutic agents for administration with the chimeric protein. Examples of such agents include, but are not limited to, agents to treat the disorders or conditions described above.

The following examples are provided solely to illustrate certain aspects of the particles and compositions that are provided herein and thus should not be construed to limit the scope of the claimed invention.

### EXAMPLES

The following EXAMPLES provide illustrative embodiments. Certain aspects of the following EXAMPLES are disclosed in terms of techniques and procedures found or contemplated by the present inventors to work well in the practice of the embodiments. In light of the present disclosure and the general level of skill in the art, those of skill will appreciate that the following EXAMPLES are intended to be exemplary only and that numerous changes, modifications, and alterations can be employed without departing from the scope of the presently claimed subject matter.

### Example 1: Trifunctionalized Nanoengagers for NK-Cell Mediated Immunotherapy

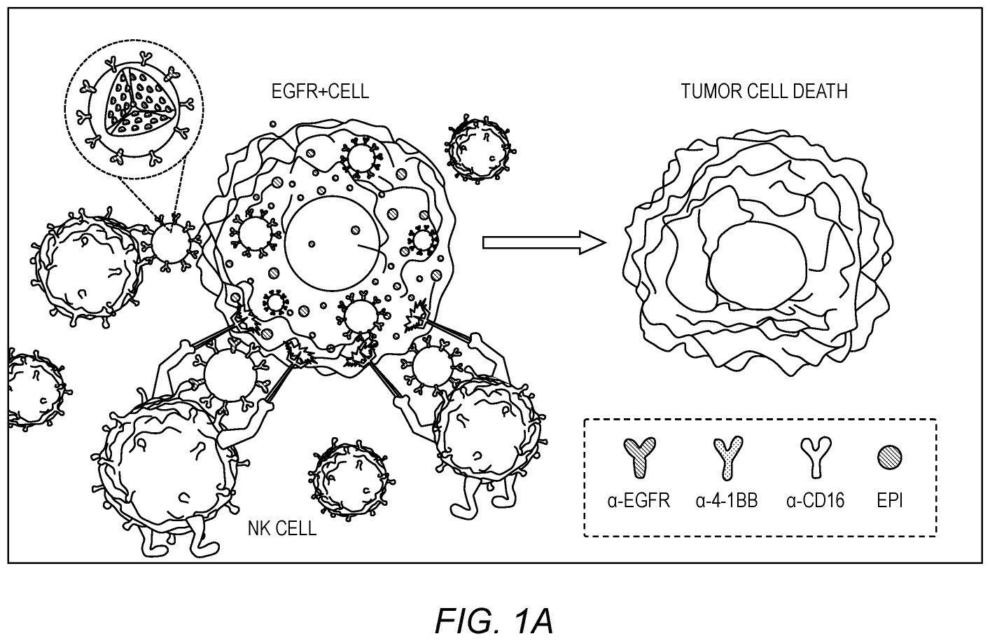

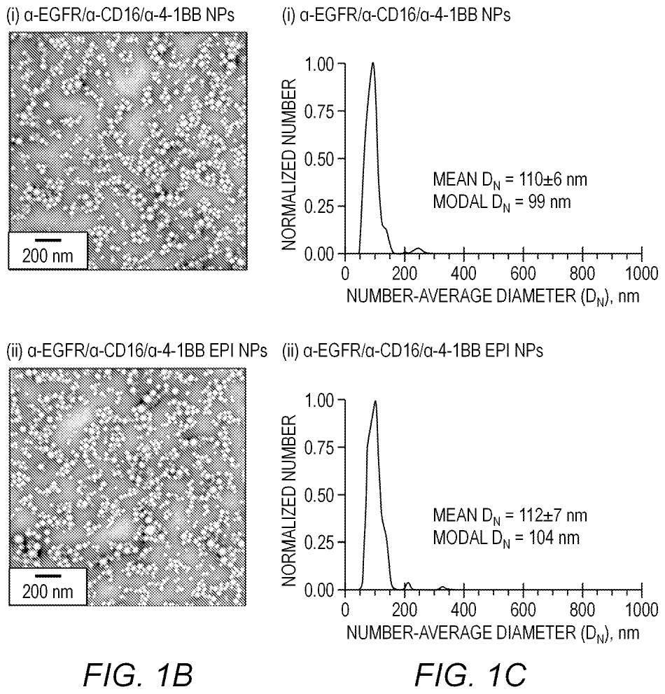

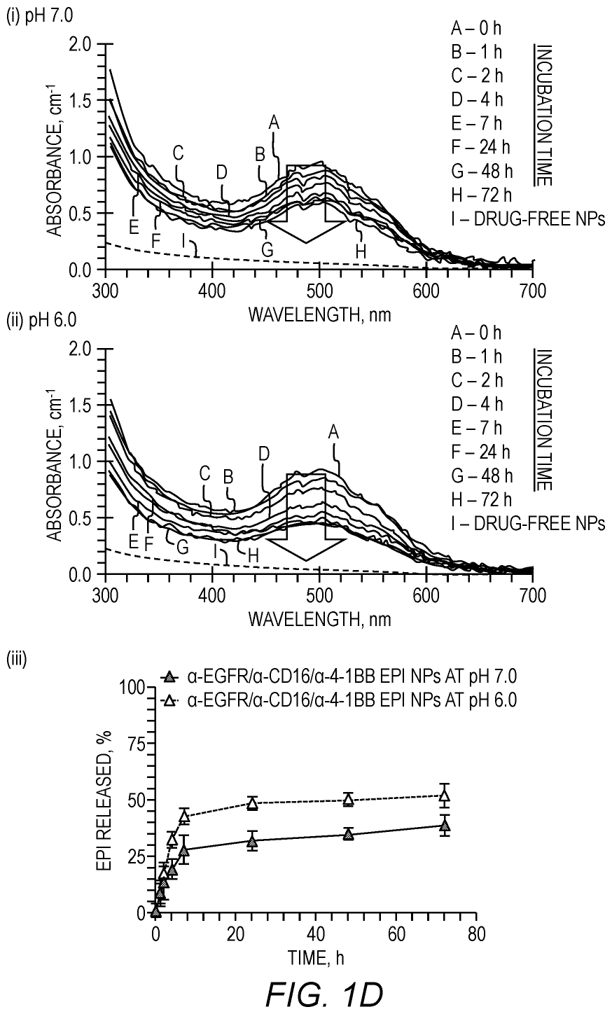

This study developed a nanoengager platform that can target epidermal growth factor receptor (EGFR)-expressing tumors and enable NK cell-mediated immunotherapy. The nanoengagers can deliver chemotherapeutics to tumors and further enhance therapeutic effects. The nanoengager platform is based on the biocompatible poly(ethylene glycol)-block-poly(lactide-co-glycolide) PEG-PLGA nanoparticle (NP). The NPs are functionalized with cetuximab (anti-human EGFR antibody, α-EGFR), and two NK activating agents: anti-CD16 (α-CD16) and anti-4-1BB (α-4-1BB) antibodies. The chemotherapeutic epirubicin (EPI) can also be encapsulated within the NPs. These trivalent nanoengagers were not only tailored for controlled-release EPI at the EGFR-overexpressed tumor but also designed to recruit and activate circulating NK cells after systemic administration (FIG. 1A).

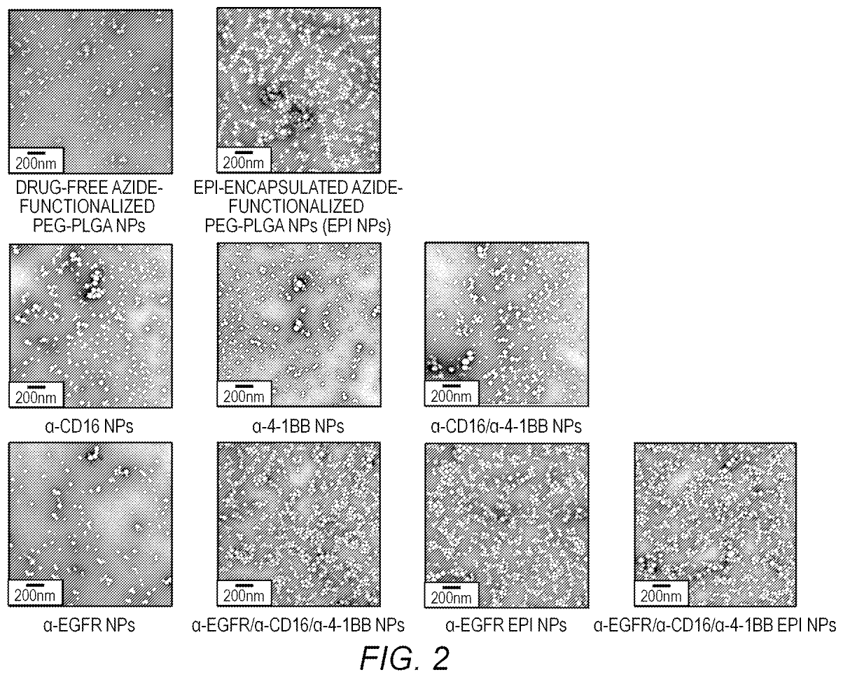

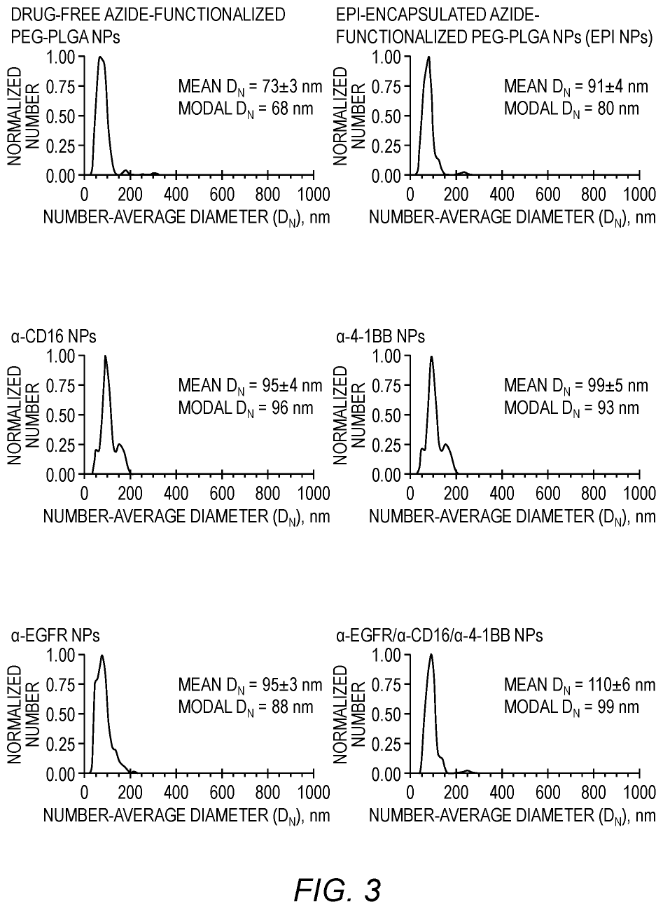

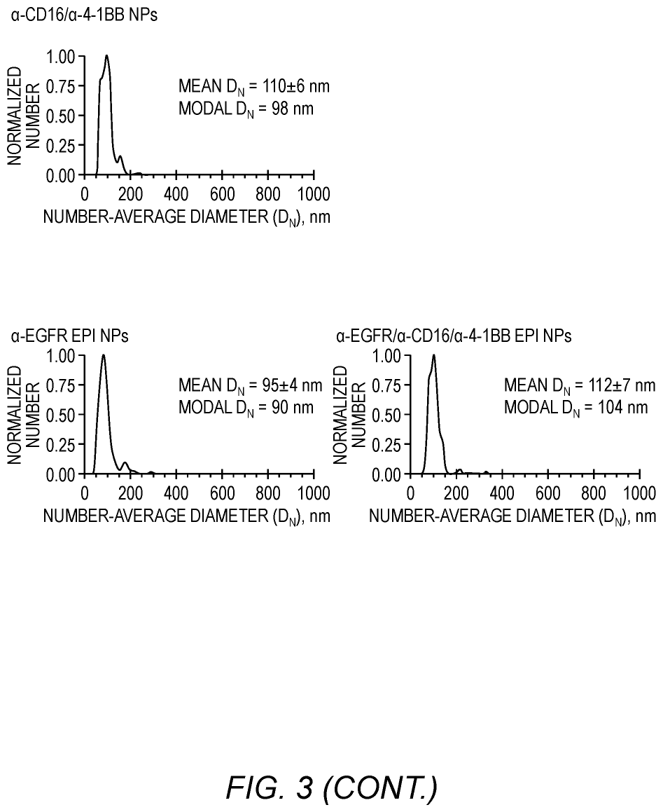

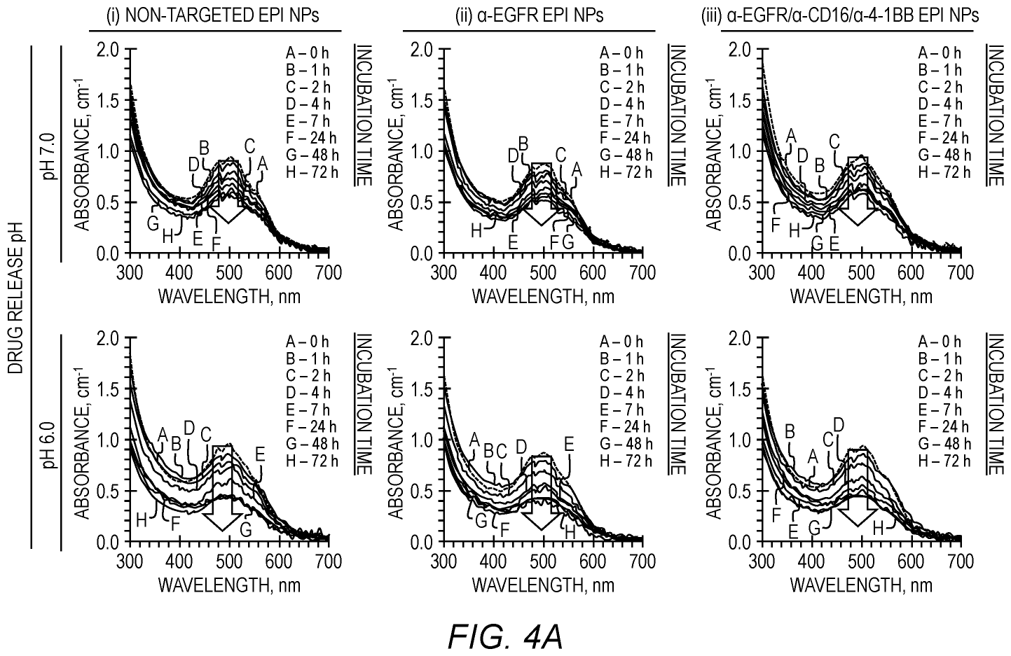

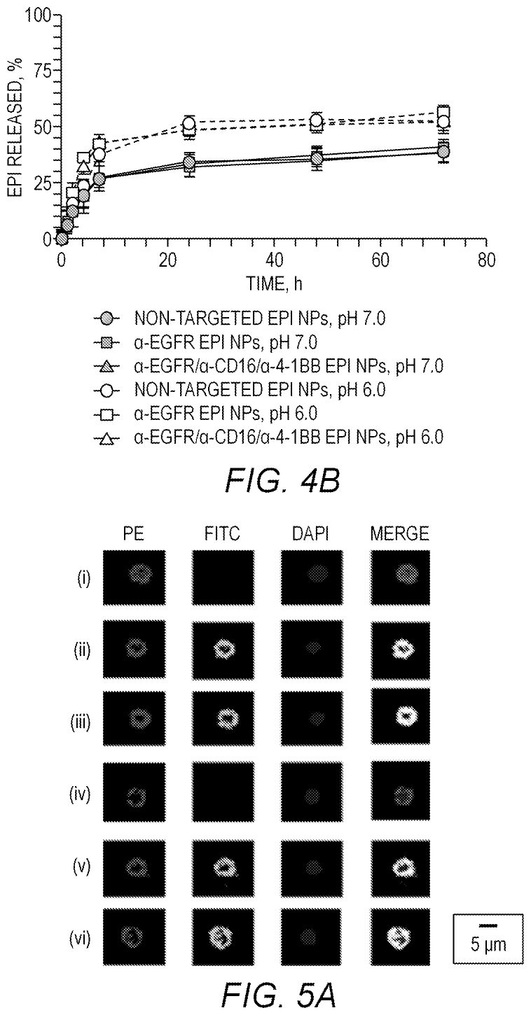

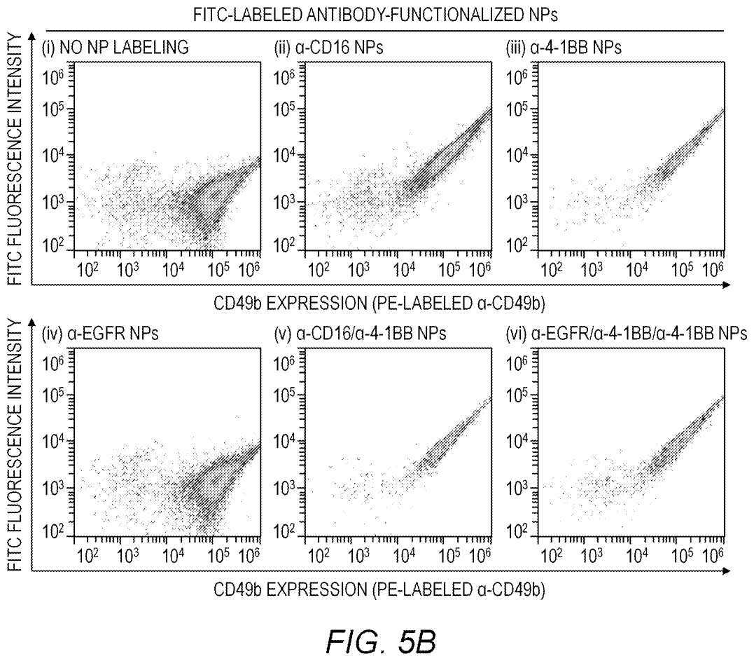

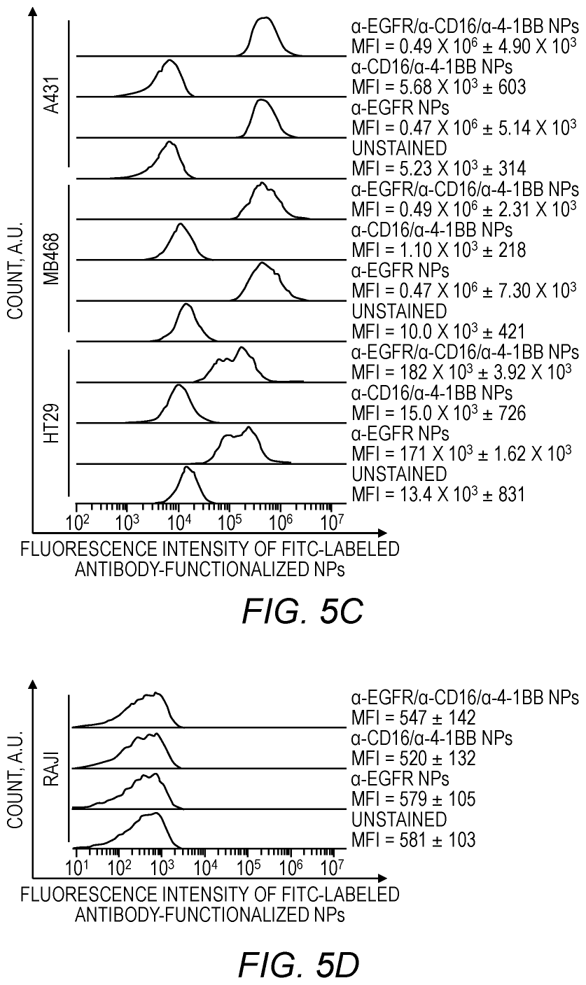

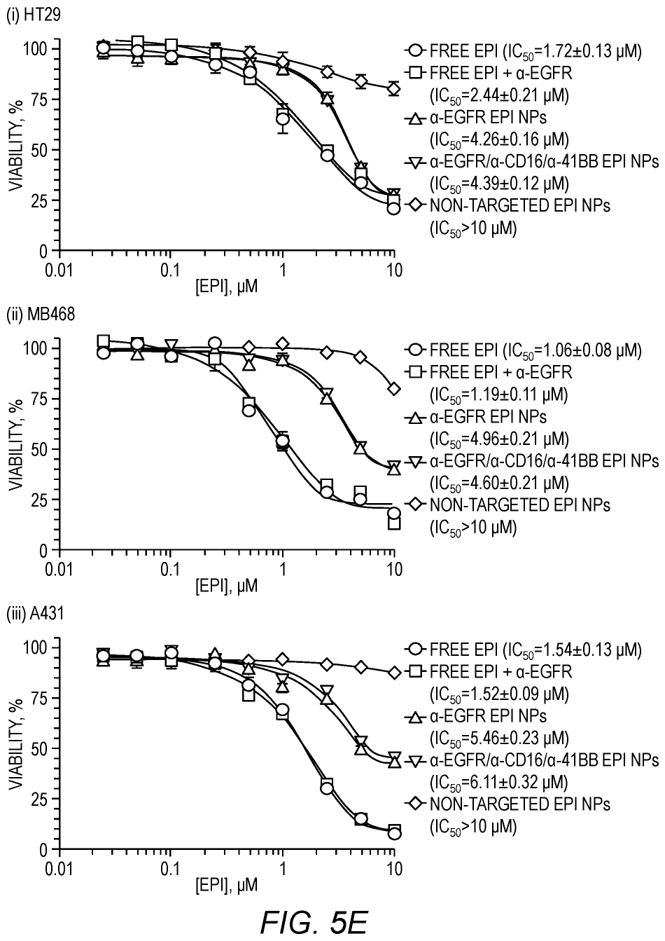

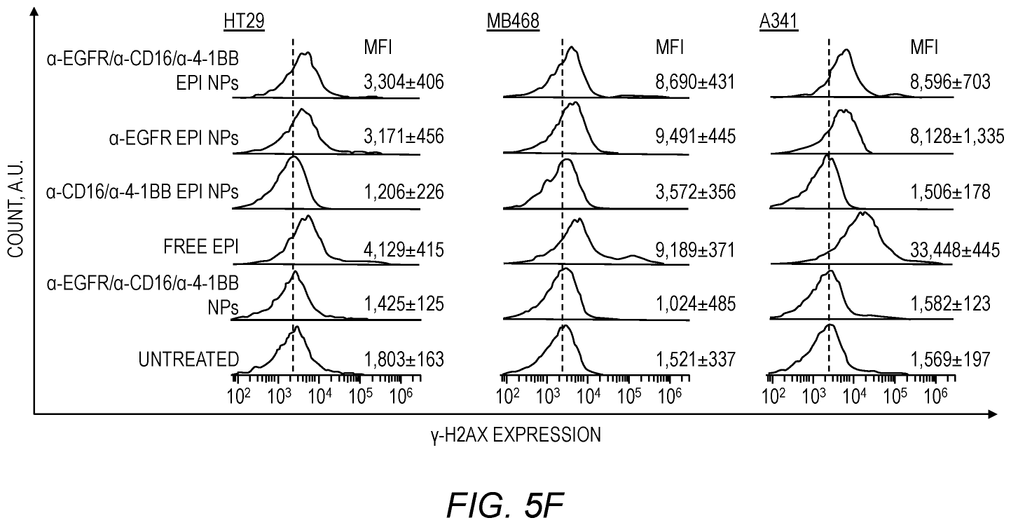

Design of multivalent EGFR-targeted nanoengagers for NK cell-mediated chemoimmunotherapy: Multivalent non-targeted and EGFR-targeted α-CD16- and α-4-1BB-functionalized drug-free and EPI-encapsulated PEG-PLGA NPs (EPI NPs) were engineered via a two-step fabrication method (FIGS. 1B, 1C, 2, and 3; Table 1). The core azide-functionalized drug-free and EPI-encapsulated NPs were first prepared via the nanoprecipitation method (Au et al. 2019 ACS Cent. Sci. 5(1):122-144). Dibenzocyclooctyne (DBCO)-functionalized α-CD16, α-4-1BB, and α-EGFR were then quantitatively conjugated to the azide-functionalized NPs via copper-free azide-cyclooctyne cycloaddition (Au et al. 2018 ACS Nano 12(2):1544-1563). A 1:1 α-CD16 to α-4-1BB molar ratio and a 1:1:1 α-CD16 to α-4-1BB to α-EGFR molar ratio were used for the fabrication of bivalent and trivalent NPs. The EPI NPs were encapsulated with approximately 2.7 wt/wt % of EPI (FIG. 1D). The encapsulated EPI underwent pH-dependent controlled release at physiological conditions, with approximately half of the encapsulated EPI released at the weakly acidic (pH 6.0) extracellular tumor microenvironment and early endosomal conditions in the first 24 h (FIG. 1D and FIGS. 4A-4B). A fluorescence-activated cell sorting (FACS) binding assay confirmed that the α-CD16- and α-4-1BB-functionalized NPs selectively bind to A488-labeled murine CD16 and Texas Red-labeled murine 4-1BB, respectively. A further in vitro binding assay and confocal laser scanning microscopy (CLSM) study confirmed that all four different FITC-labeled multivalent α-CD16 and/or α-4-1BB NPs bind selectively to the NK cells (FIGS. 5A-5B). The binding affinities of different α-EGFR-functionalized NPs to EGFR-overexpressed HT29 (colorectal adenocarcinoma), MB468 (triple-negative breast cancer), and A431 (epidermoid carcinoma) cells were verified by an in vitro binding assay (FIG. 5C) and CLSM. No nonspecific binding was observed in the control EGFR non-expressing Raji cells (FIG. 5D). An in vitro CLSM study confirmed that all three EGFR-overexpressed cancer cells took up the encapsulated EPI after brief incubation with the EGFR-targeted NPs. The targeted EPI-encapsulated NPs showed direct anticancer activities against the HT29, MB468, and A431 cells with a half-maximal inhibitory concentration (IC₅₀) of between 4 and 6 (FIG. 5E), whereas the same concentrations of non-targeted EPI NPs or NP-anchored antibodies showed insignificant toxicities. The γ-H2AX assay (FIG. 5F) confirmed the formation of DNA double-strand breaks in the cancer cells as a result of the intercalation of EPI into cell DNA.

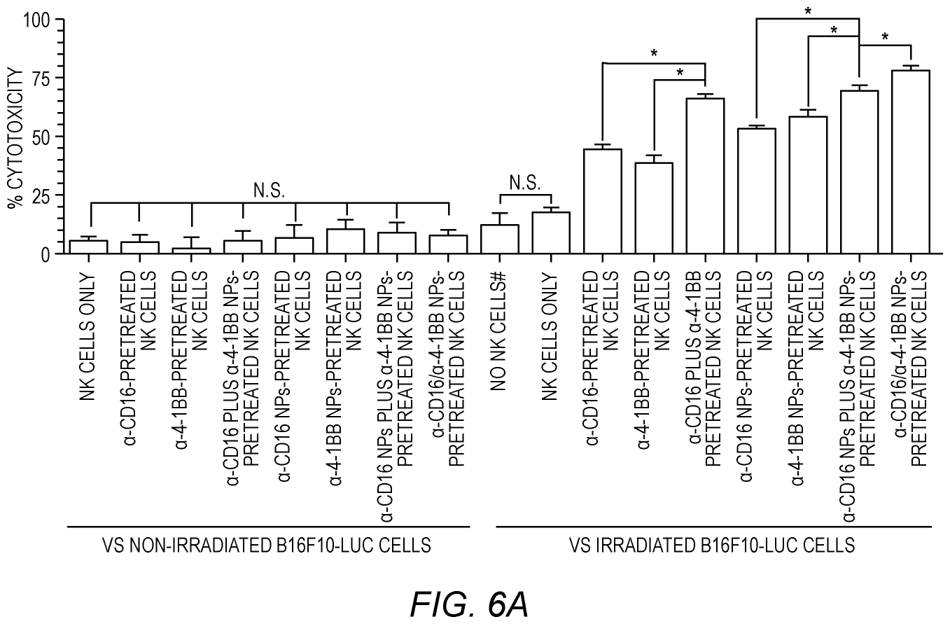

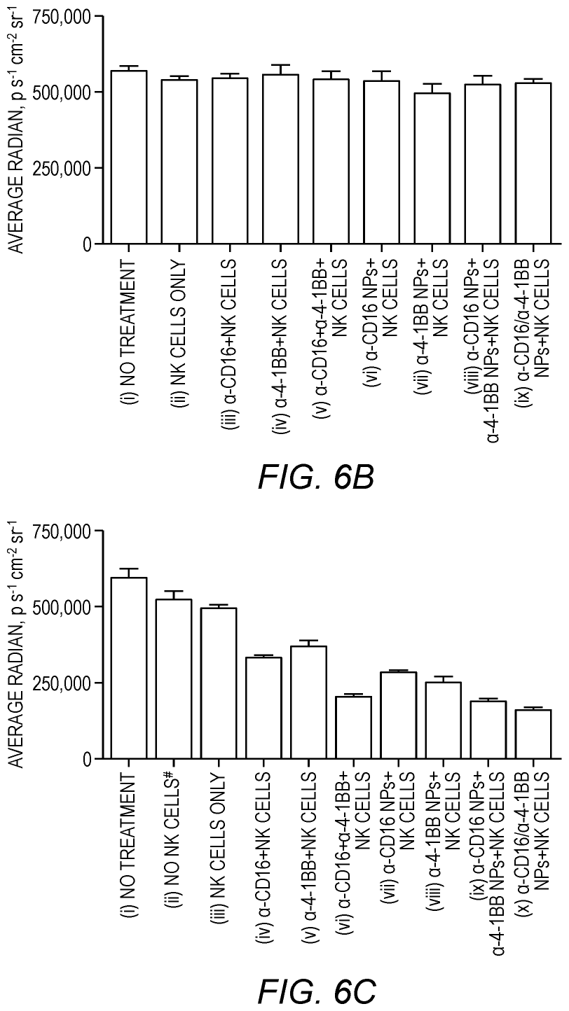

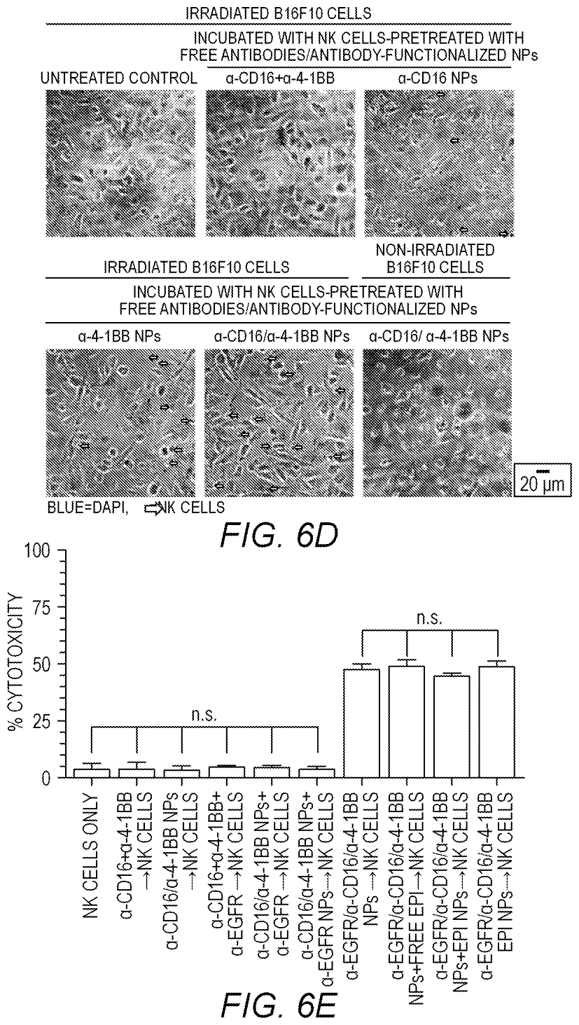

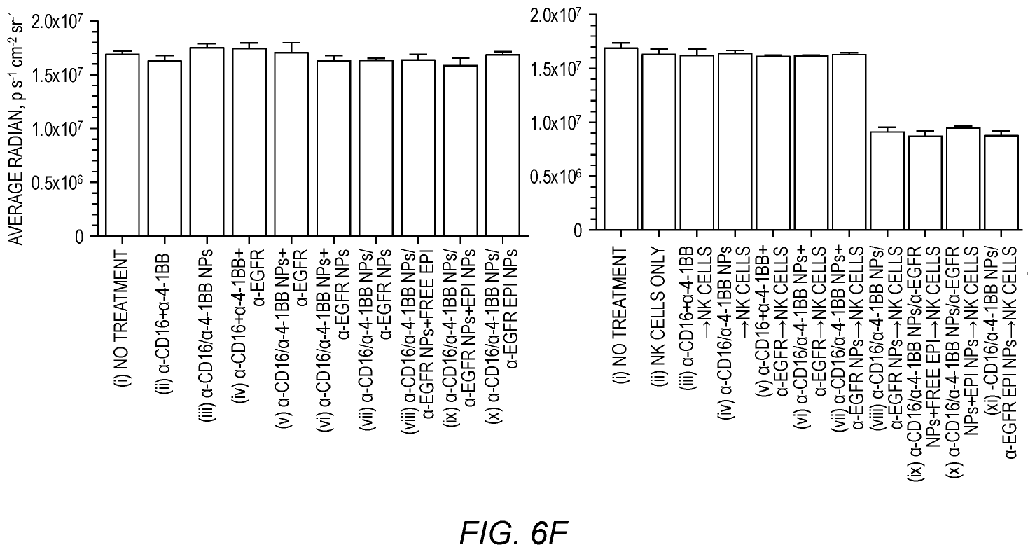

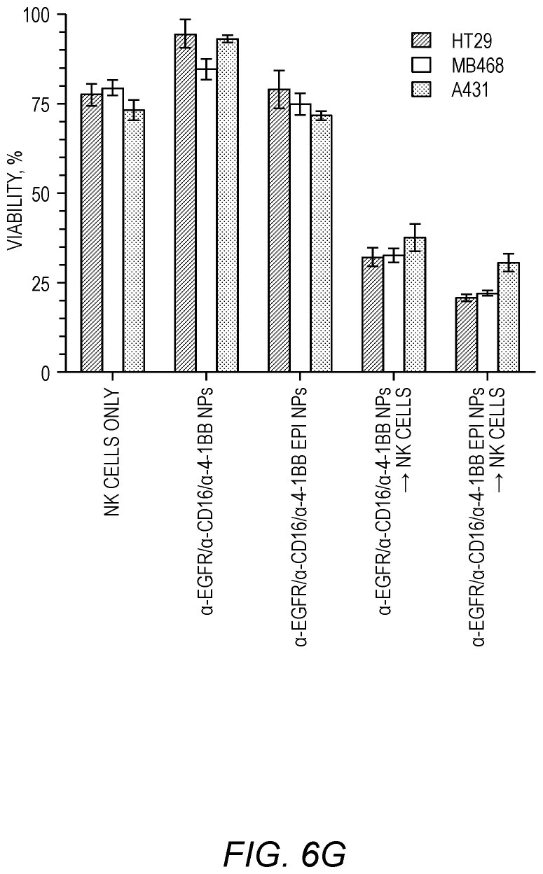

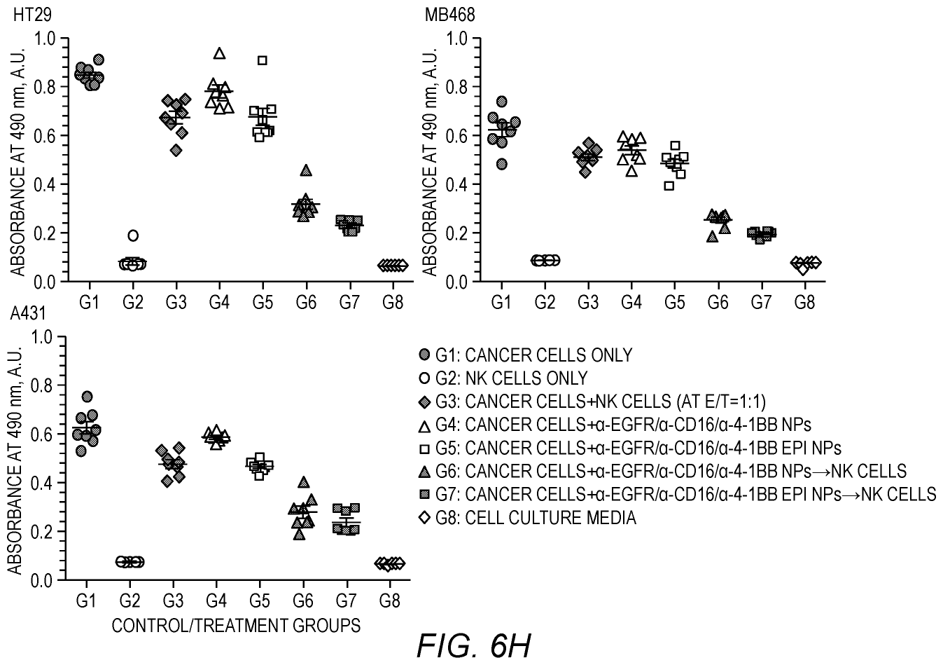

α-CD16 and α-4-1BB-functionalized nanoparticles can effectively activate NK cells in vitro: One goal of this study was show that the NP formulation of α-CD16 and α-4-1BB is more effective at NK activation than free α-CD16 and α-4-1BB antibodies. To demonstrate that the effective spatiotemporal activation of CD16 and 4-1BB stimulatory molecules on NK cells can increase NK cell-mediated specific lysis, an NK cell cytotoxicity assay was performed in the presence of luciferase-labeled B16F10 (B16F10-Luc) targeted cells. NK cells alone showed limited direct cytotoxicity (about 10%) against B16F10-Luc cells at a 1:1 effector/target (E/T) ratio (FIGS. 6A-6B) as the NK cells did not recognize/bind to the cancer cells and were not activated. To enable cancer cell recognition or binding, tumor cells were given 5Gy irradiation (IR) to upregulate NK cell-activating ligands (e.g., CD112, ULBP-1) on the surface of B16F10-Luc cells. Upon this immune stimulation, NK cells showed moderate cytotoxicity against B16F10-Luc cells (FIG. 6A). Pretreating NK cells with free α-CD16 or α-4-1BB significantly increased the cytotoxicity to 44.4±2.6% and 38.0±3.7% (FIG. 6A and FIG. 6C), respectively. α-CD16 NPs- and α-4-1BB NPs-pretreated NK cells showed significantly higher toxicities (52.7±1.9% and 57.9±3.5%, respectively) than free antibody-pretreated NK cells. This increased cytotoxicity can be explained by increased cooperative binding and more effective ligation (“clustering”) of the CD16 and 4-1BB co-stimulatory molecules by the NPs. Most importantly, NPs containing both NK activating agents (α-CD16/α-4-1BB NP) pretreatment further increased the NK cell cytotoxicity to 77.1±2.1%, which is significantly higher than pretreatment with free α-CD16 plus free α-4-1BB (p=0.0019 versus treatment) and α-CD16 NPs plus α-4-1BB NPs (p=0.0207). The increased cytotoxicity can be explained by the simultaneous activation of both stimulatory molecules and the clustering effect in the dual-antibody-functionalized NPs that cannot be achieved by combining both free agonistic antibodies. The engagement of α-CD16/α-4-1BB NPs-pretreated NK cells with the immunostimulated B16F10 cells was directly confirmed by phase-sensitive optical microscopy (FIG. 6D).

Next, it was investigated how the EGFR-targeted trifunctionalized nanoengagers improve NK cell cytotoxicity against the firefly luciferase-expressing HT29 cells (HT29-Luc2). Similar to the B16F10-Luc cells, NK cells alone showed very low cytotoxicity against the HT29-Luc2 cells (FIG. 6F, top panel). Similarly, HT29-Luc2 cells pretreated with free α-CD16 and α-4-1BB or α-CD16 NPs and α-4-1BB NPs in the presence of free α-EGFR or α-EGFR NPs did not significantly affect NK cell cytotoxicity as the targeting ligand was not associated with the NK activating agents. On the other hand, both drug-free and EPI-encapsulated trifunctional nanoengagers (α-EGFR/α-CD16/α-4-1BB NP) significantly increased NK cell cytotoxicity (FIG. 6E and FIG. 6F, bottom panel). This increase in therapeutic efficacy is attributed to the targeting effect of α-EGFR as well as its linkage to NK activating agents. In this study, the EPI did not significantly affect NK cell cytotoxicity (FIG. 6E). Further in vitro toxicity studies confirmed that a sub-therapeutic dose of drug-free or EPI-encapsulated trivalent nanoengagers can effectively enhance the cytotoxicity of NK cells against the HT29, MB469, and A431 cells (FIGS. 6G and 6H). The enhancement of NK cell cytotoxicity could not be achieved by the combination of free α-EGFR, α-CD16, and α-4-1BB antibodies. Phase-sensitive optical microscopy study confirmed the engagement of NK cells to the α-EGFR/α-CD16/α-4-1BB NPs-pretreated cancer cells, but no significant NK cell engagement was observed in the α-CD16/α-4-1BB NPs and α-EGFR NPs-pretreated cancer cells. Therefore, the conjugated α-EGFR is essential for the trivalent NPs to recruit and activate the NK cells.

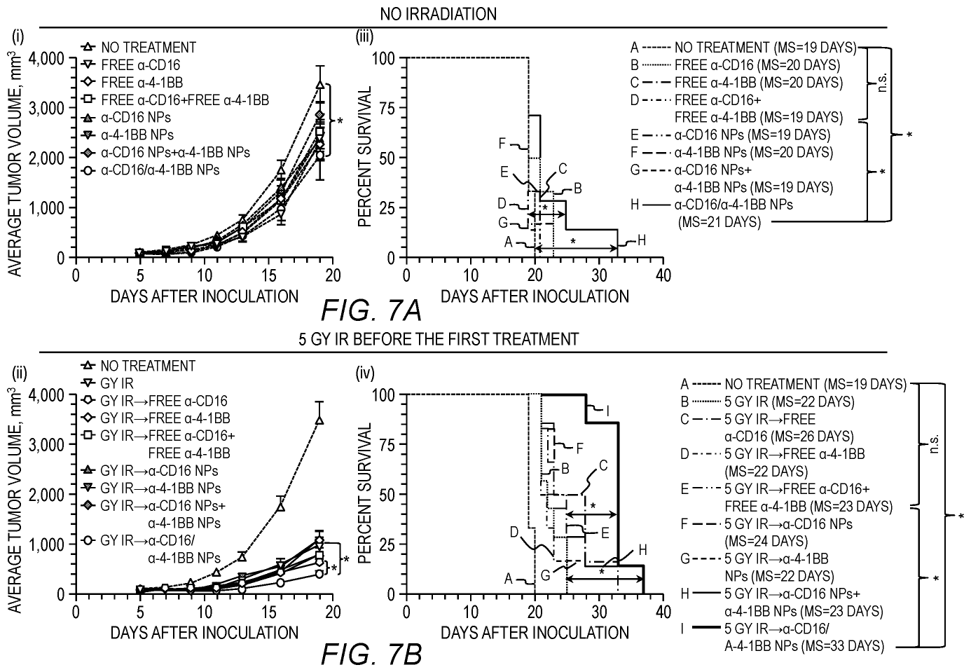

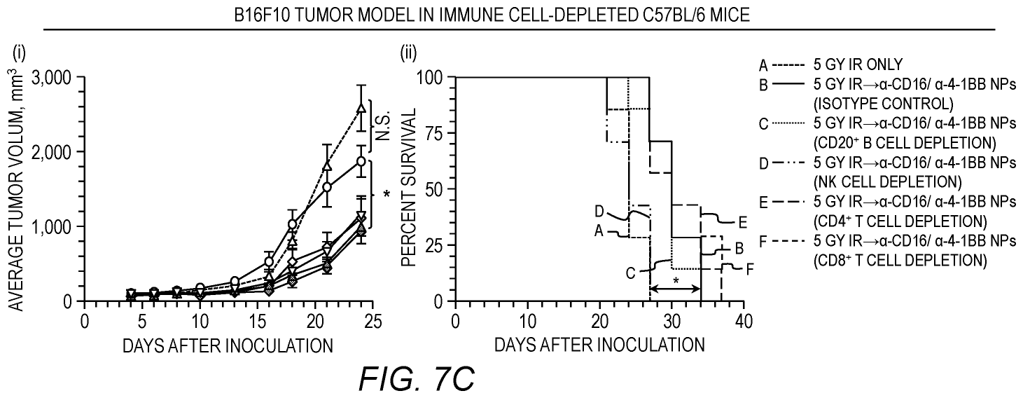

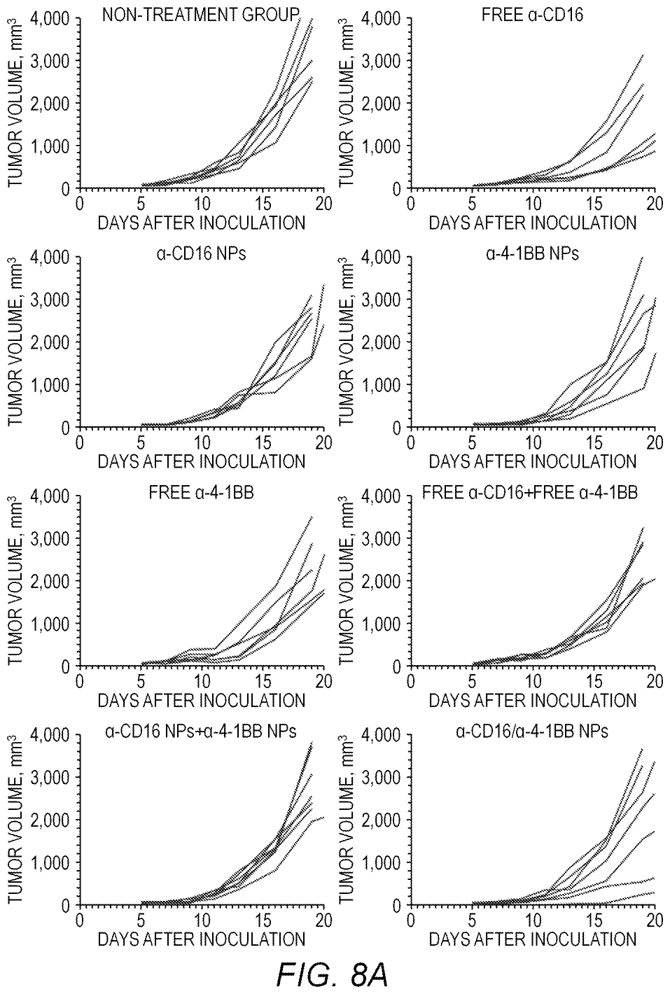

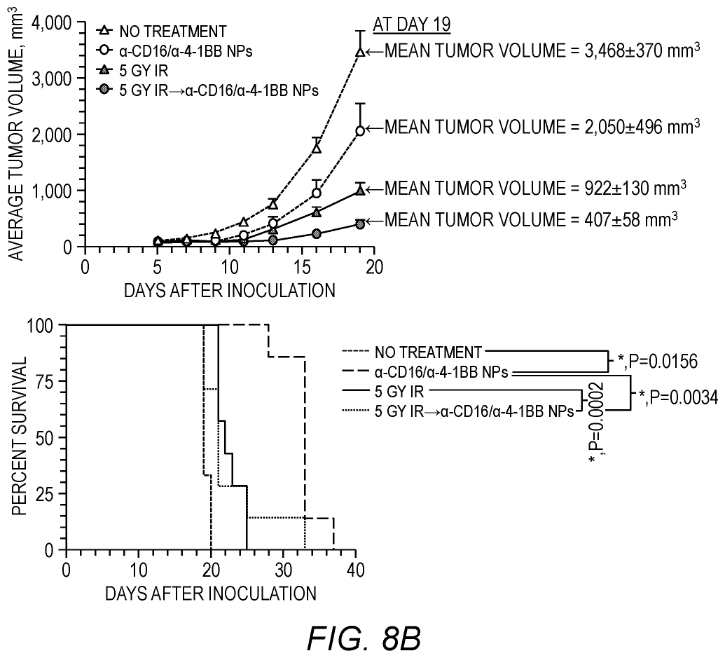

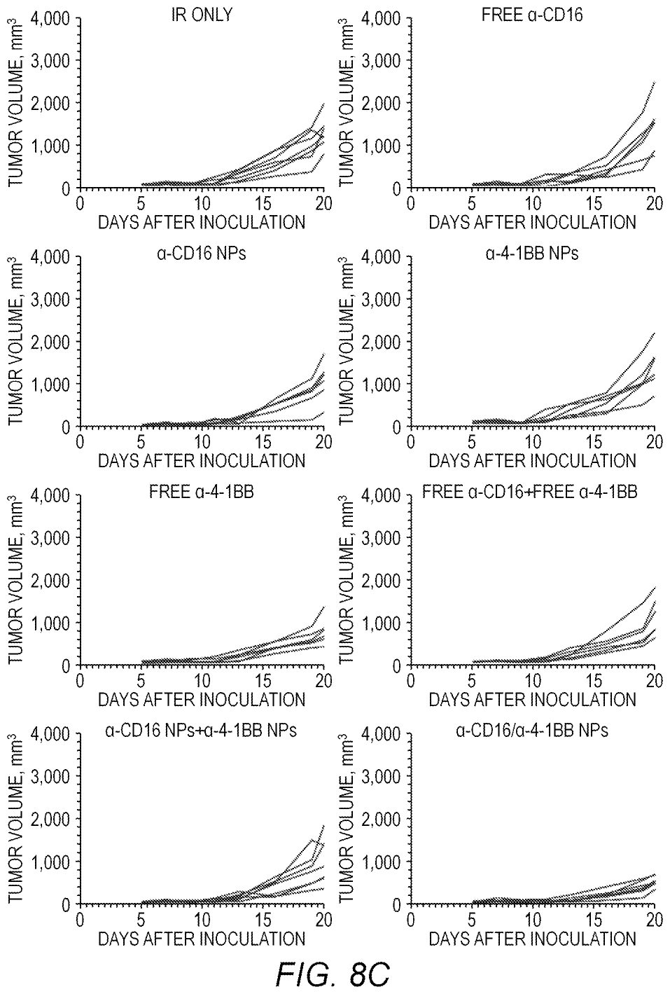

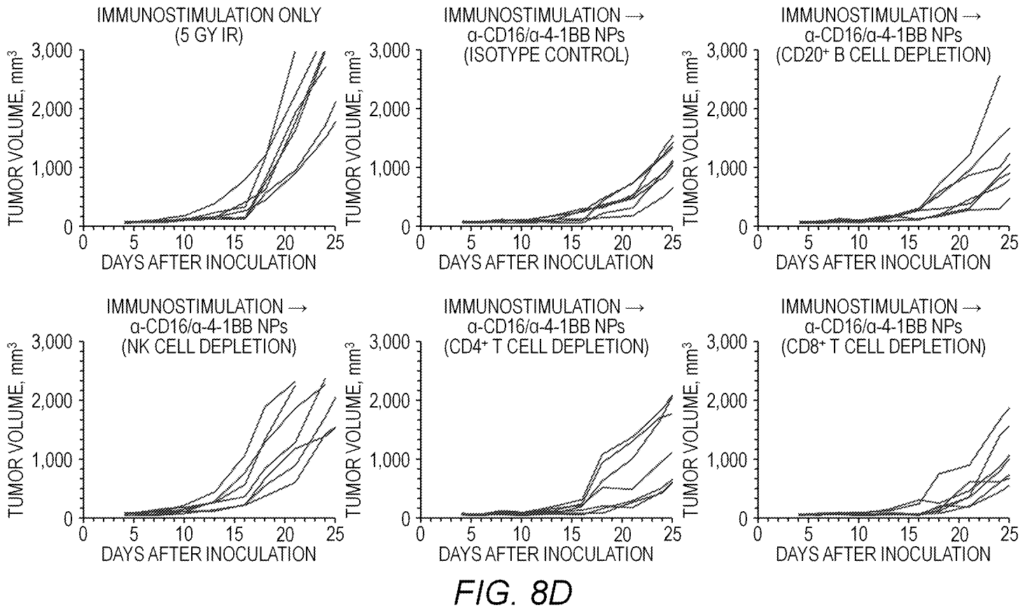

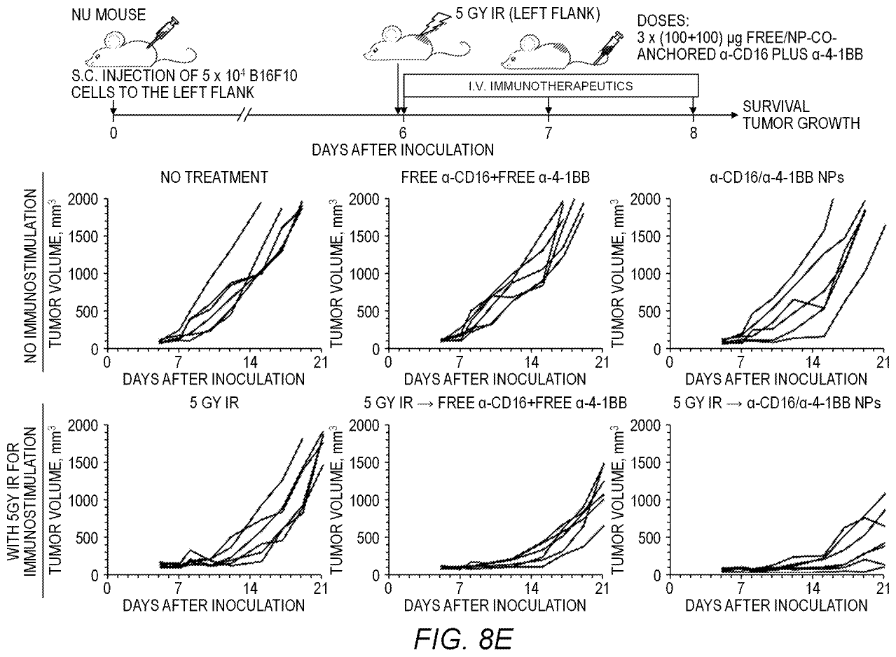

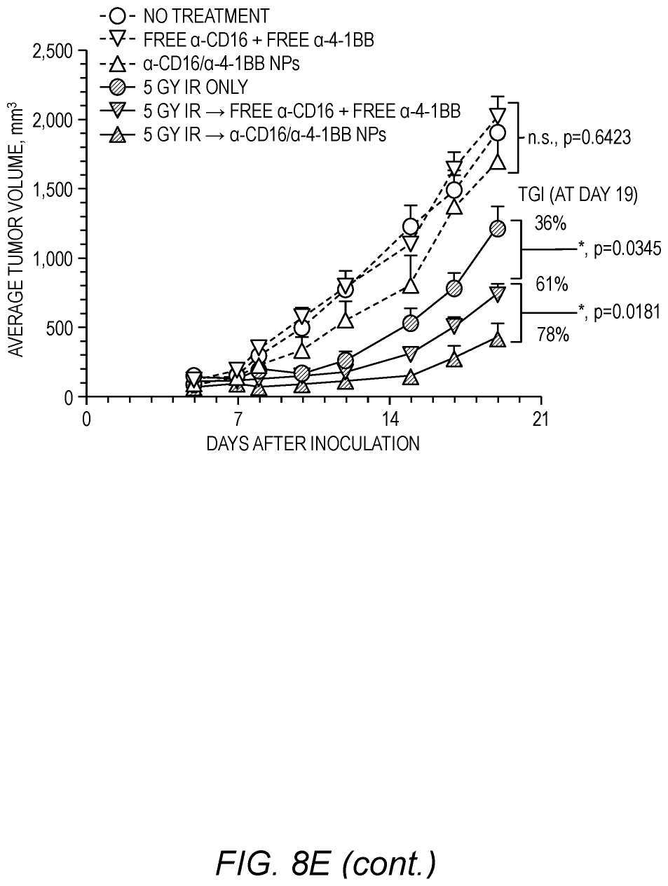

Spatiotemporal co-activation of CD16 and 4-1BB stimulatory molecules can effectively activate NK cells to eradicate cancer in vivo but requires biological targeting: The in vitro observations were validated using four mouse models of cancer. To examine the in vivo efficacy of α-CD16/α-4-1BB NPs, the B16F10 syngeneic mouse melanoma model was utilized, wherein immunocompetent C57BL/6 mice are immune cell-depleted of B cells, NK cells, CD4⁺ T cells, and CD8⁺ T cells by i.p. injection of α-CD20, a-NK1.1, α-CD4, and α-CD8 (300 μg/injection) at 5, 8, 10, 12, 15, and 18 days post-inoculation. Mouse IgG 2a (300 μg/injection) was administered as an isotype control. α-CD16/α-4-1BB NPs (containing 100 μg of each antibody) were i.v. administered at 6, 7, and 8 days post-inoculation. Immunotherapeutics contained 100 μg of α-CD16 and/or 100 μg of α-4-1BB (free or NP-conjugated) were tail vein i.v. administered at 6, 7, and 8 days post-inoculation. The xenograft tumors of mice in the immunostimulation groups were subjected to a single 5 Gy irradiation 4 h before the administration of immunotherapeutics to upregulate the NK cell-activating ligands in the cancer cells. It was found that α-CD16/α-4-BB NPs showed moderated anticancer activity (average tumor volume 40% smaller than non-treatment group at 19 days post-inoculation; p=0.0479 versus the non-treatment group) and slightly prolonged survival (absolute growth delay (AGD)=+3 days; p=0.0156 versus the non-treatment group; FIGS. 7A, 8A, and 8B). Moreover, treatments with free antibody, antibody-functionalized NPs, or their 1:1 combination did not show significant anticancer activities (FIGS. 7B and 8B). This lack of efficacy is consistent with the lack of recognition/binding of NK cells to tumor cells. The effect of α-CD16/α-4-BB NPs is likely facilitated by the nonspecific activation of NK cells throughout the animals' system. However, such system activation would be undesirable from a toxicity standpoint.

To enable NK recognition of tumor cells/targeting, tumors were irradiated with 5 Gy. Following radiation, the mice were treated with α-CD16/α-4-BB NPs or control treatments with α-CD16, α-4-BB, α-CD16 NPs, α-4-BB NPs, or their 1:1 combination. Robust treatment response with α-CD16/α-4-BB NPs was observed with tumor growth reduction of ˜60% when compared to mice that received radiotherapy only (at day 19 post-inoculation) (FIG. 7B and FIGS. 8B-8C). The combination of α-CD16 NPs and α-4-1BB NPs also inhibited tumor growth but the inhibition was less significant than α-CD16/α-4-1BB NPs. Other treatments did not significantly delay tumor growth when compared to control. These findings suggest that both effective NK activation and tumor targeting/binding are all essential mechanisms in NK cell-mediated cancer treatment.

Since CD16 and 4-1BB can also activate the adaptive immune system in syngeneic models, an immune cell depletion study was performed in the B16F10 tumor model to validate that the treatment effects are due to NK cell activities. The depletion of CD20⁺ B cells, CD4⁺ T cells, and CD8⁺ T cells did not significantly affect the anticancer efficacy of α-CD16/α-4-1BB NPs (p=0.4448, 0.5590, and 0.4859 versus the isotype control group, respectively; FIG. 7C and FIG. 8D). On the other hand, the depletion of NK cells by α-NK1.1 significantly reduced the anticancer efficacy of the α-CD16/α-4-1BB NPs (p<0.0001 versus the isotype control group). These therapeutics were also examined in a B16F10 xenograft tumor model in T cell-deficient athymic nude (Nu) mice. These mice lack adaptive immune systems, and α-CD16/α-4-1BB NPs (targeted by radiotherapy) was an effective treatment (FIG. 8E), further confirming that the mechanism of action of these NPs is through the innate immune system.

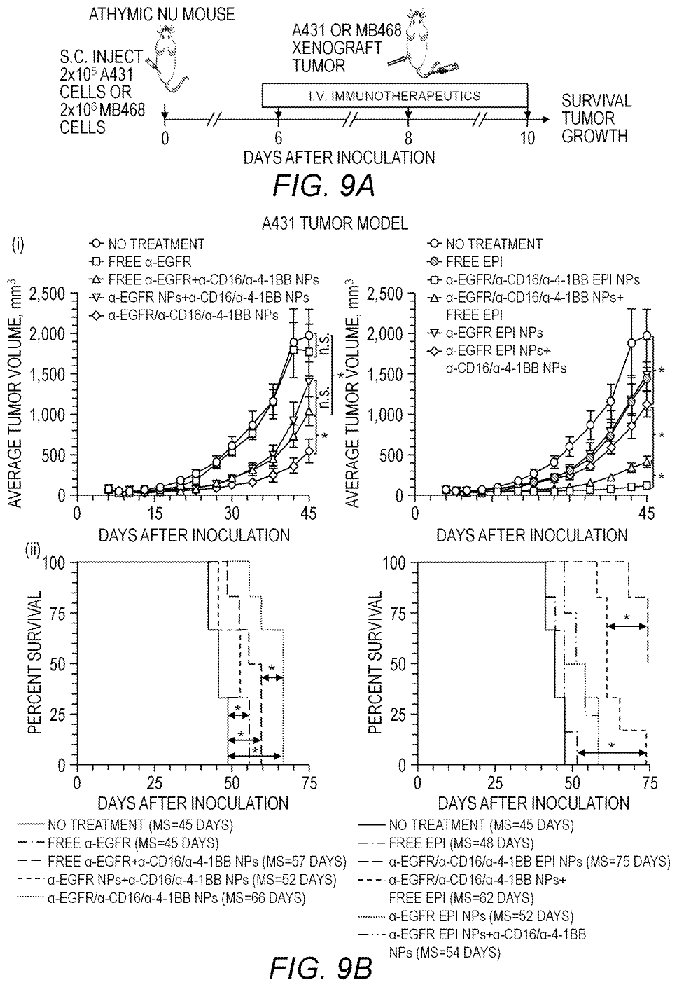

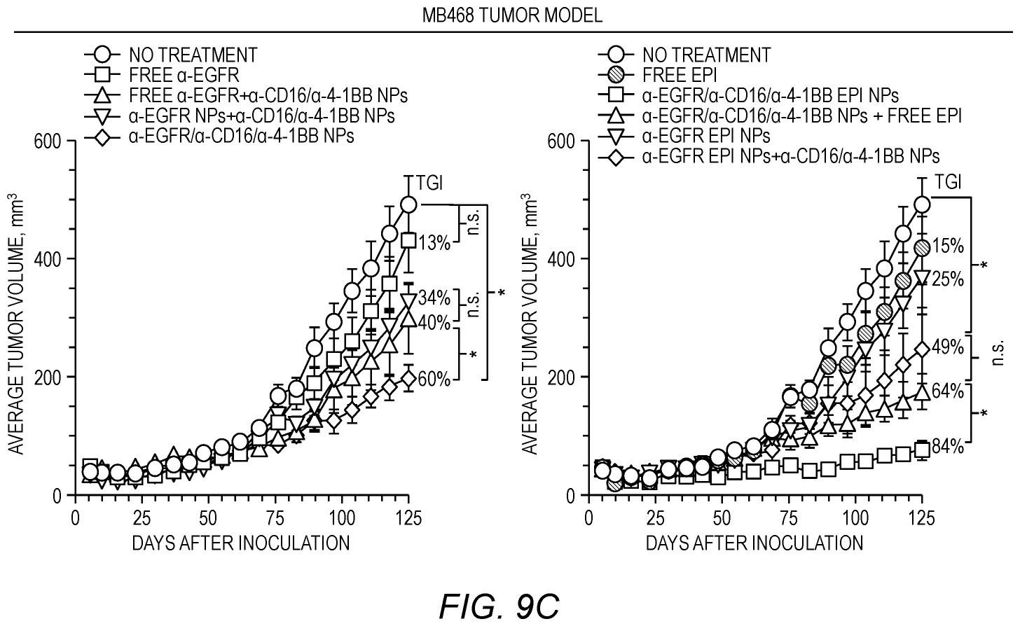

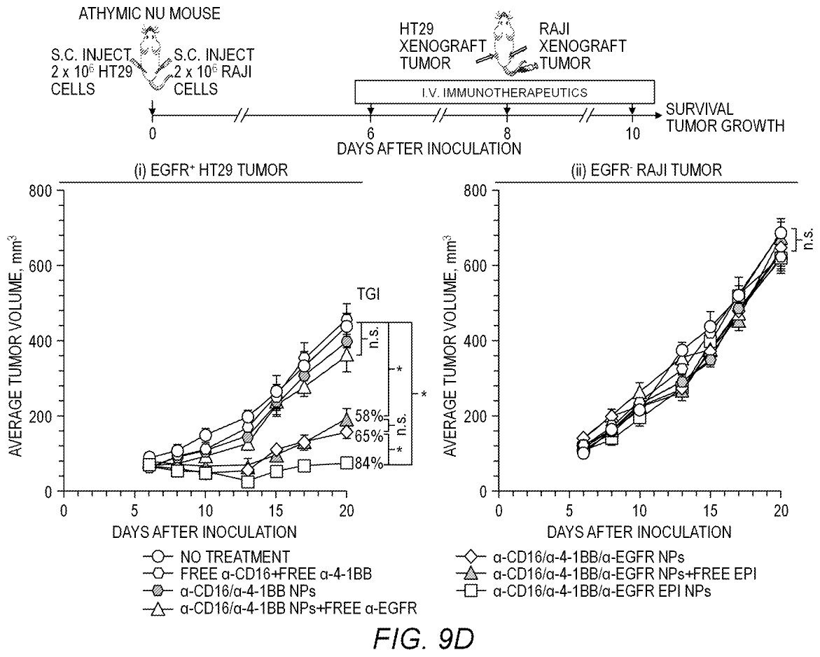

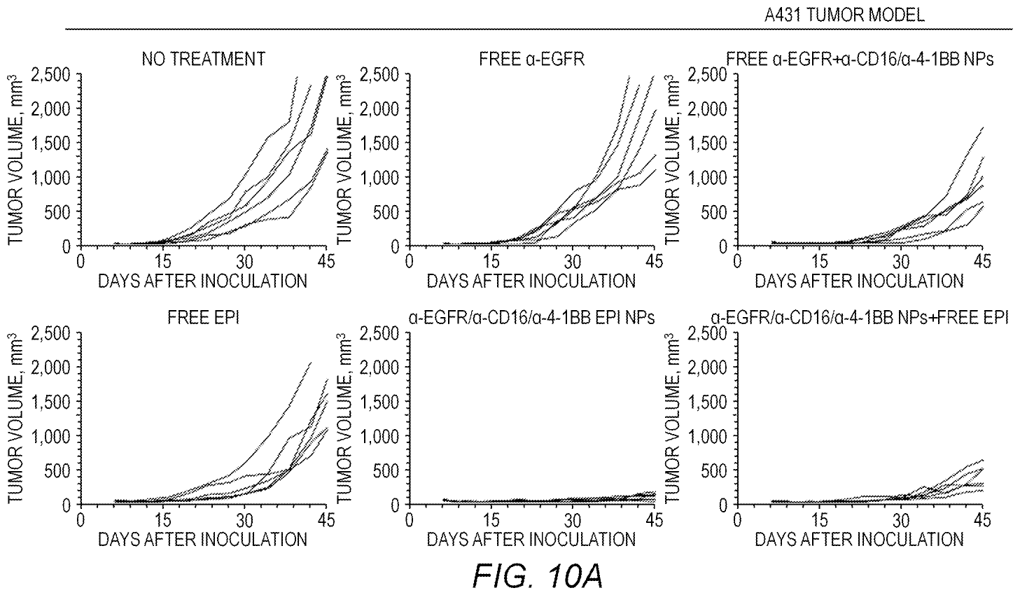

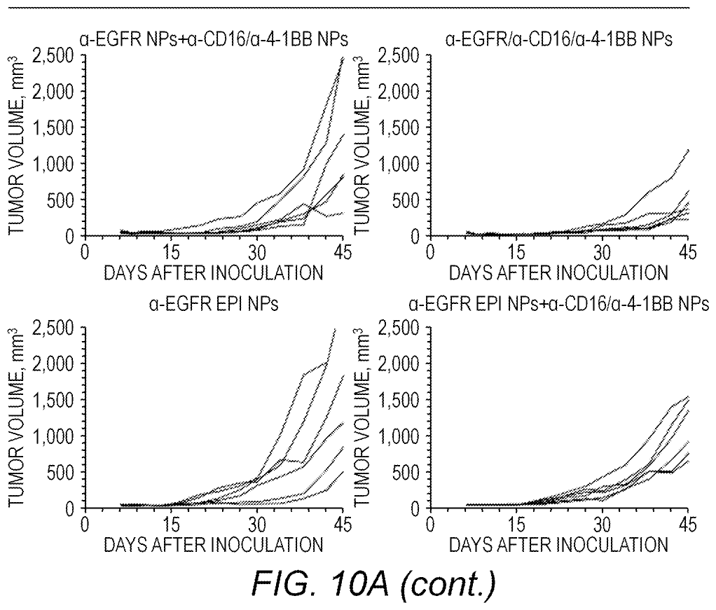

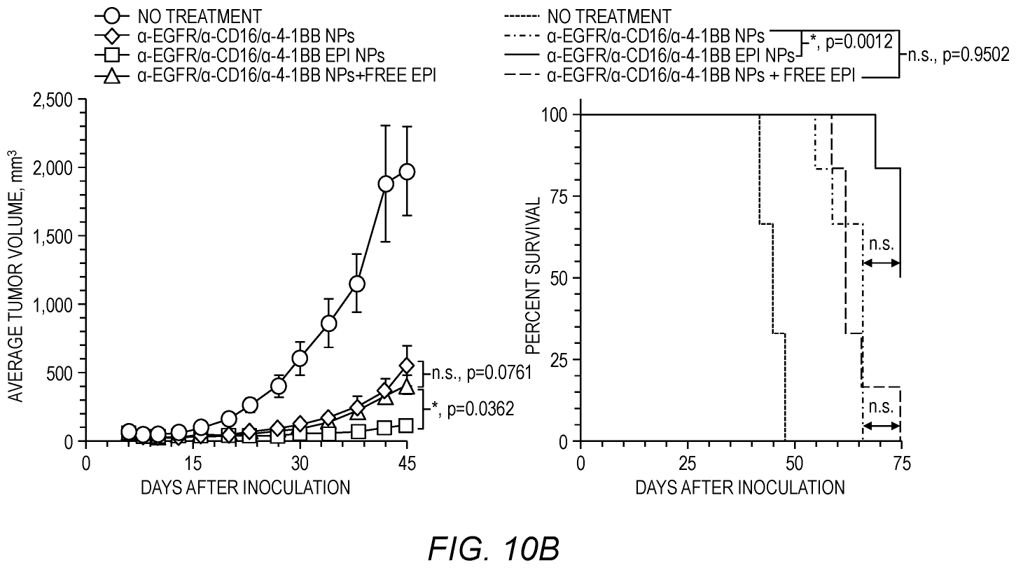

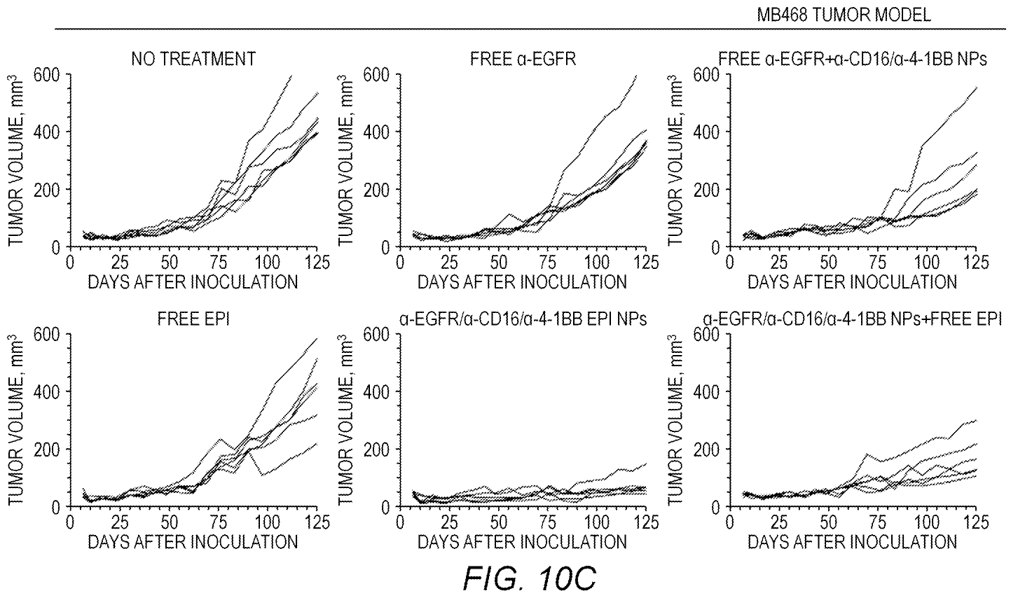

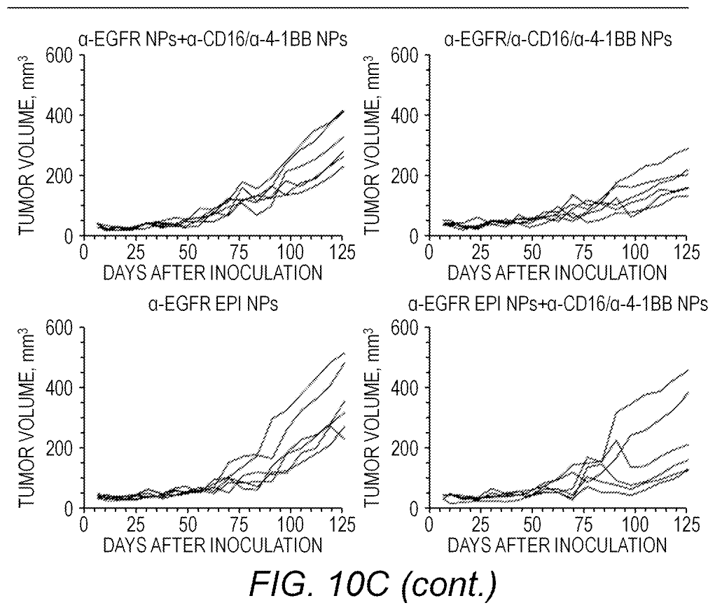

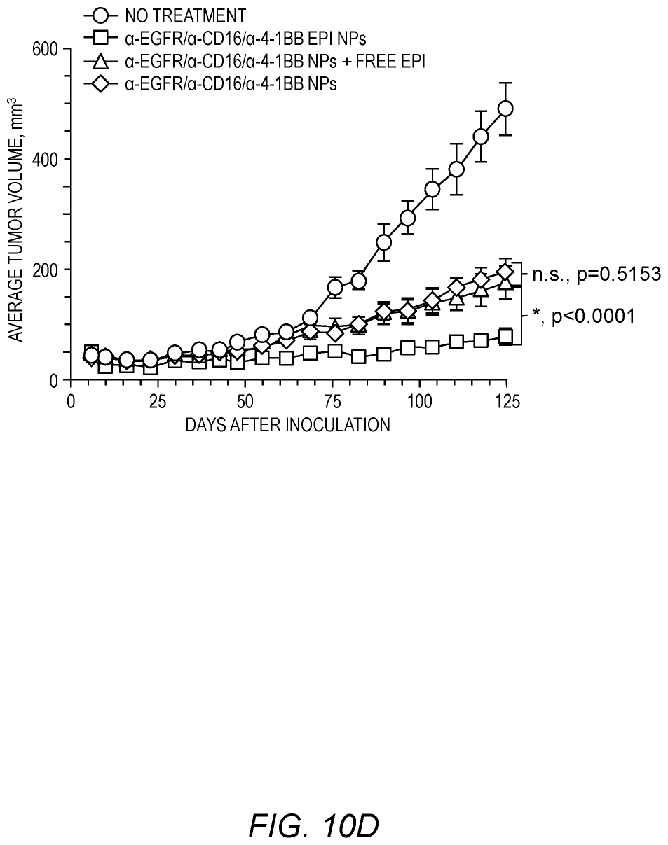

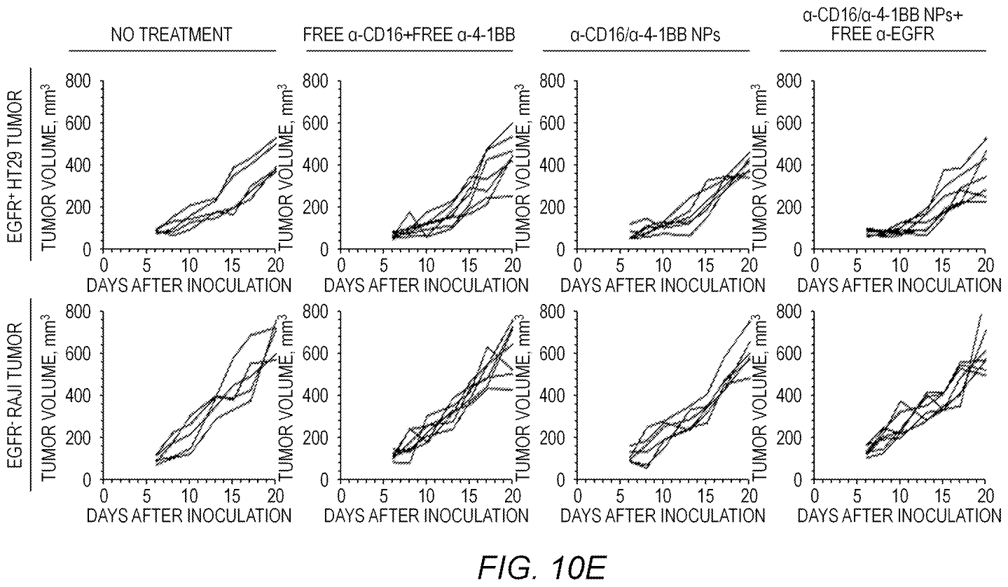

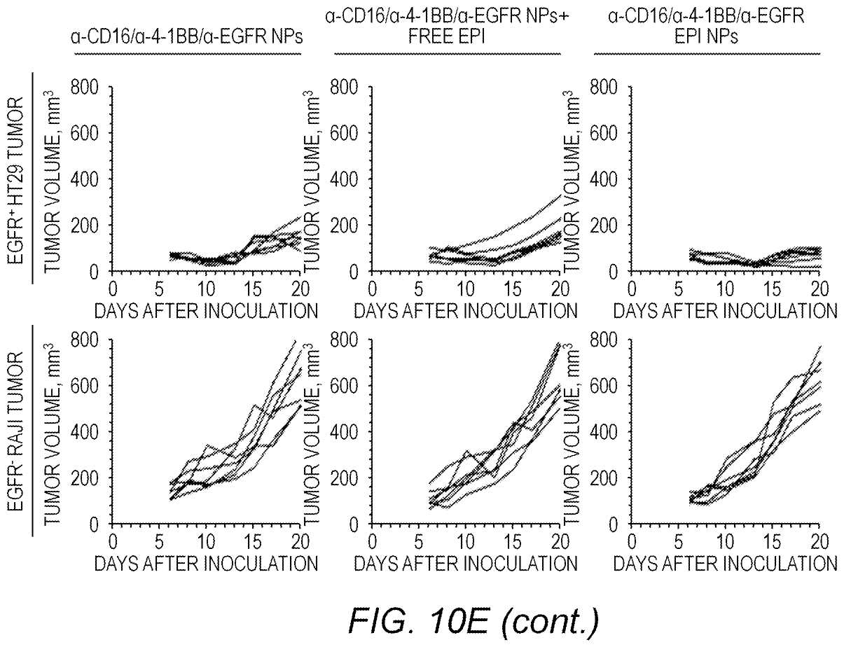

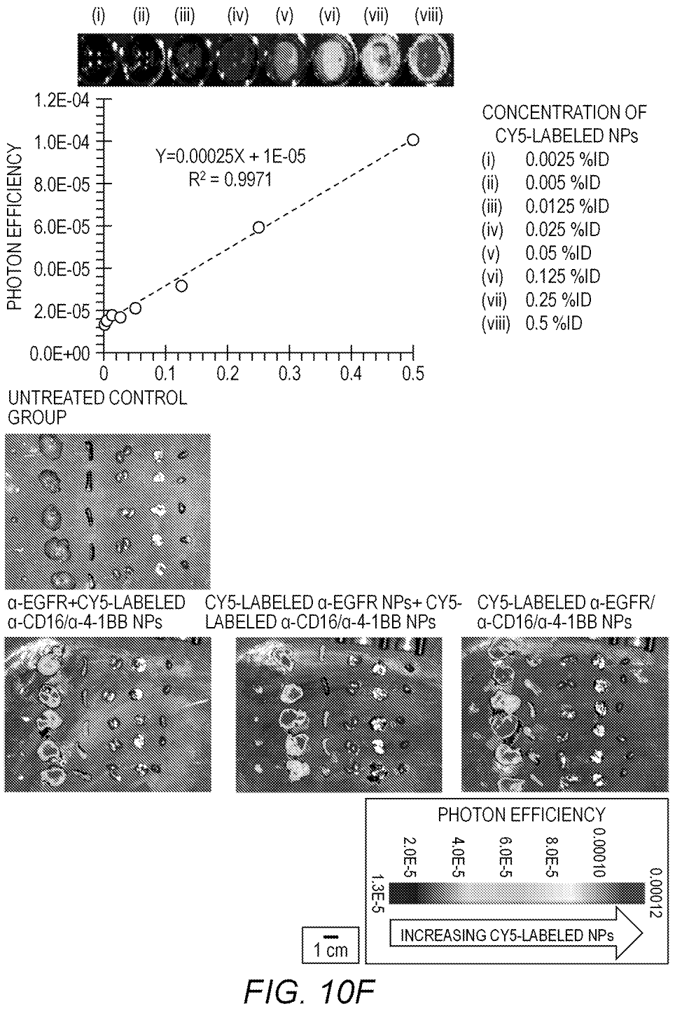

EGFR-targeted trifunctionalized nanoengagers effectively inhibit EGFR-overexpressed cancer growth in vivo: Given that radiotherapy cannot be utilized to target systemic disease, this study aimed to engineer nanoengagers that can target tumor cells through a targeting ligand. EGFR targeting was chosen to demonstrate the proof of principle. To demonstrate that the EGFR-targeted trivalent nanoengagers allow effective NK-cell mediated immunotherapy and chemoimmunotherapy without further external immunostimulation, a comprehensive in vivo anticancer efficacy study was performed in the EGFR-overexpressed A431 tumor model (FIG. 9A). This study showed that EGFR targeting alone does not confer an effective treatment, with α-EGFR treatment showing a minimal effect when compared to the control (p=0.6127 versus non-treatment group; FIGS. 9B and 10A). The treatment with free α-EGFR and α-CD16/α-4-1BB NPs or α-EGFR NPs and α-CD16/α-4-1BB NPs led to moderate delays in tumor growth (p=0.0046 and 0.0061 versus the non-treatment group, respectively). The treatment with α-EGFR/α-CD16/α-4-1BB NPs had the most robust treatment responses with tumor growth delays averaging 24 days after the initial treatment and prolonged survival averaging 18 days compared to the non-treatment group (p=0.0018). This data confirmed that the EGFR-targeted nanoengagers can effectively guide NK cells to attack the EGFR-overexpressed tumor cells without needing external stimulation.

Since NPs can also deliver chemotherapeutics and enable chemoimmunotherapy, the use of EGFR-targeted nanoengagers with a chemotherapy payload was examined. EPI was used as a model drug. The anticancer activities of free EPI, α-EGFR EPI NPs, α-EGFR/α-CD16/α-4-1BB EPI NPs, α-EGFR/α-CD16/α-4-1BB NPs and free EPI, and α-CD16/α-4-1BB NPs and α-EGFR EPI NPs were compared. Treatments with free EPI and α-EGFR EPI NPs slightly reduced the rate of tumor growth (p=0.0017 and p=0.0061 versus the non-treatment group, respectively; FIGS. 9B and 10A). Chemoimmunotherapy with α-EGFR/α-CD16/α-4-1BB NPs and free EPI administered separately did not significantly improve the efficacy compared to α-CD16/α-4-1BB/α-EGFR NPs alone (p=0.8531; FIGS. 9B and 10B). However, the EGFR-targeted chemoimmunotherapy with α-EGFR/α-CD16/α-4-1BB EPI NPs effectively inhibited the tumor growth for approximately 40 days and significantly prolonged survival (p=0.0017 and 0.0362 versus the non-treatment group and treatment with α-EGFR/α-CD16/α-4-1BB NPs and free EPI, respectively. At the study endpoint (75 days post-inoculation), half of the mice treated with α-EGFR/α-CD16/α-4-1BB EPI NPs were alive, while none of the mice in the other treatment groups achieved long-term survival. This result highlights that effective targeted chemoimmunotherapy can only be achieved when the chemotherapeutics and agonistic antibodies reach the cancer at the same time.

To confirm these findings, an in vivo efficacy study was conducted in an MB468 tumor model to validate the anticancer effect of both drug-free and EPI-encapsulated nanoengagers (FIG. 9A). Similar to the anticancer activity observed in the A431 tumor model, treatment with the drug-free α-EGFR/α-CD16/α-4-1BB NPs significantly slowed the tumor growth (p=0.0002 versus the non-treatment group) and resulted in tumor growth inhibition (TGI) of 60% (FIGS. 9C and 10C). Chemoimmunotherapy with α-EGFR/α-CD16/α-4-1BB EPI NPs showed robust anticancer activity against the MB468 tumor, with 83% of the treated mice having stable disease (i.e., less than 25% increase in tumor volume) at the study endpoint (TGI=84%). On the other hand, treatment with α-EGFR/α-CD16/α-4-1BB NPs plus free EPI or α-EGFR EPI NPs plus α-CD16/α-4-1BB NPs only slowed the tumor growth rate and resulted in TGIs of 64% and 49%, respectively (p<0.0001 versus the treatment with α-EGFR/α-CD16/α-4-1BB EPI NPs; FIGS. 9C and 10D). This indicates that encapsulating the chemotherapeutics into EGFR-targeted nanoengagers enhances the effectiveness of targeted concurrent chemoimmunotherapy.

To further validate the importance of tumor targeting in NK cell-based treatment, the nanoengagers were examined using a dual-xenograft tumor model with EGFR expressing HT29 tumors and EGFR negative Raji tumors (FIG. 9D). The EGFR-negative Raji tumor model was chosen as a negative control because it is sensitive to NK cell-mediated lysis and insensitive to small-molecule anthracycline treatment, given the overexpression of the multidrug resistance protein 1 receptor. Similar to the A431 and MB468 tumor models, treatment with free α-CD16 and α-4-1BB, α-CD16/α-4-1BB NPs, and α-CD16/α-4-1BB NPs plus free α-EGFR did not inhibit the growth of the HT29 tumor (p=0.1171 versus the non-treatment group) and the Raji tumor (p=0.1171 versus the non-treatment group; FIGS. 9D and 10E) because NK cells did not recognize the tumors. On the other hand, EGFR-targeted immunotherapy with α-EGFR/α-CD16/α-4-1BB NPs significantly delayed HT29 tumor growth and resulted in 66% TGI at the study endpoint (p=0.0081 versus the non-treatment group; FIGS. 9D and 10F). However, this treatment did not significantly affect the Raji tumor growth (p=0.2805 versus the non-treatment group; FIGS. 9D and 10E). The data from this study firmly establish that biological targeting is critical to NK-mediated immunotherapy, and EGFR-targeted nanoengagers are highly effective and specific to EGFR-expressing tumors. Similar to the anticancer activity observed in the A431 and MB468 tumor models, co-treatment with free EPI plus α-EGFR/α-CD16/α-4-1BB NPs did not further improve the treatment effect of the HT29 tumor (p=0.2014 versus treatment with α-EGFR/α-CD16/α-4-1BB NPs; FIGS. 9D and 10E). Conversely, treatment with the α-EGFR/α-CD16/α-4-1BB EPI NPs completely inhibited HT29 tumor growth and resulted in an average TGI of 84% (p=0.0113 versus the non-treatment group, p=0.0276 versus treatment with α-CD16/α-4-1BB/α-EGFR NPs plus free EPI). The improved anticancer activity against the HT29 tumor can be explained by the targeted delivery of EPI to the EGFR-overexpressed tumor. Raji tumor growth was not affected by this targeted treatment (p=0.0503 versus the non-treatment group). Although HT29 has a lower EGFR antigen expression than A431 and MB468, the lack of efficacy observed in the EGFR-negative Raji tumor in all treatment groups confirmed that the observed antitumor activity involved specific engagement between the targeted cancer cells and NK cells rather than the systemic activation of the innate immune system.

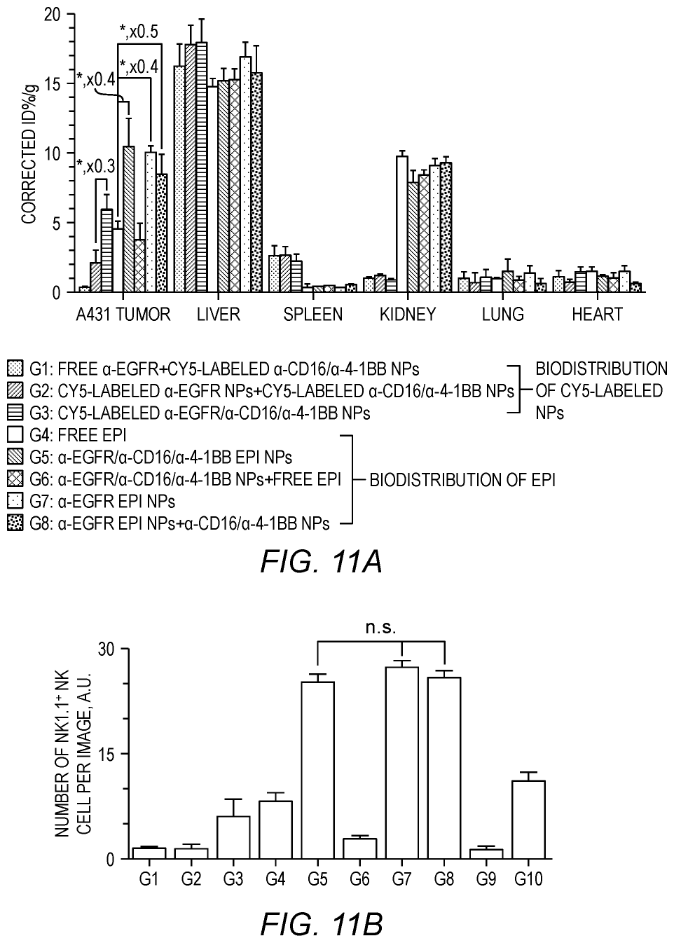

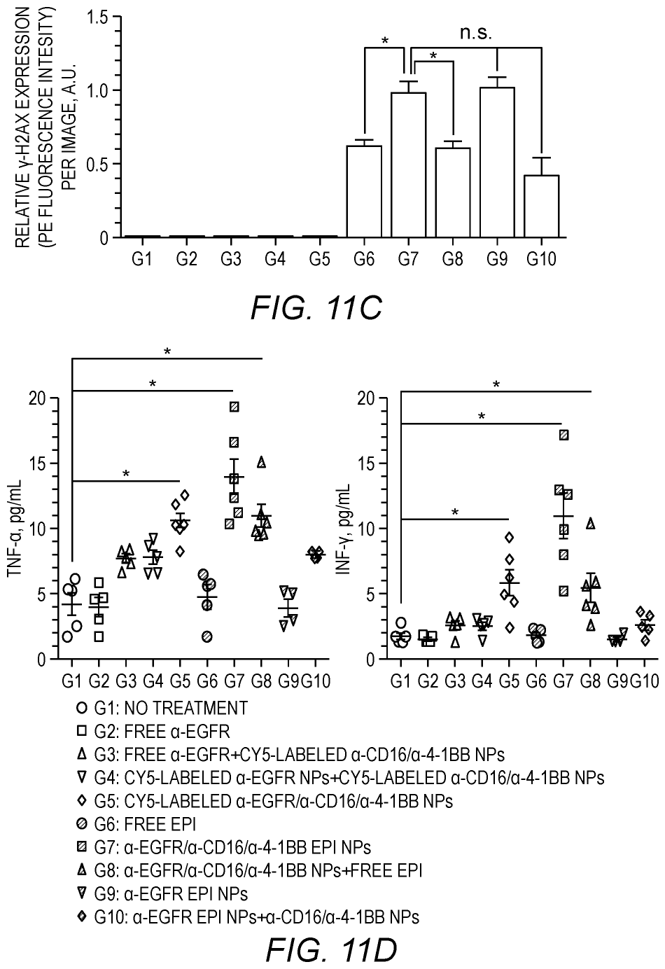

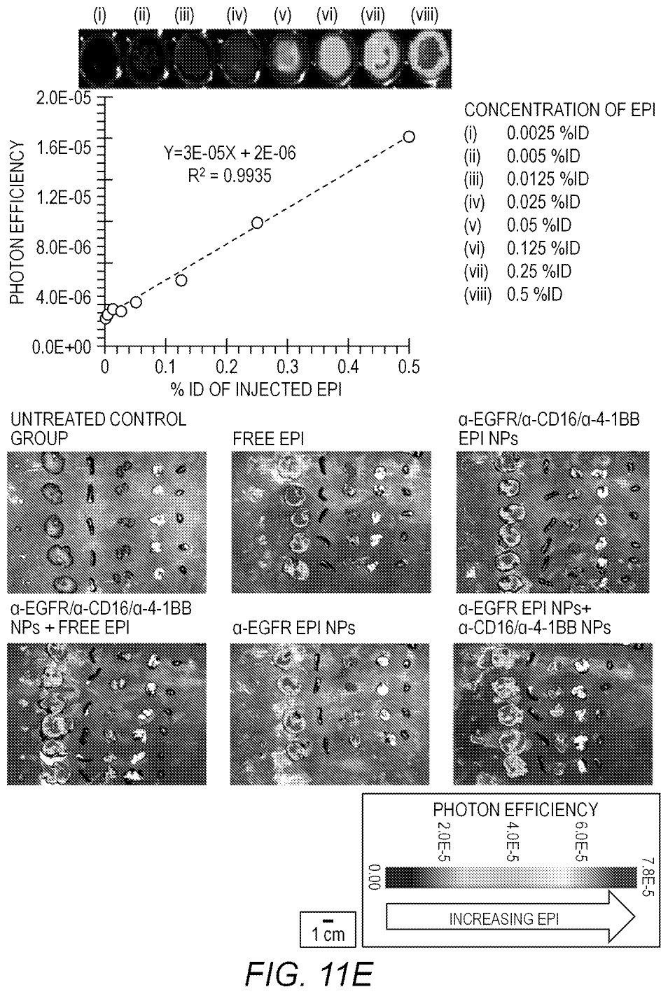

Mechanistic insight into the EGFR-targeted trifunctionalized nanoengagers for NK cell-mediated chemoimmunotherapy: To gain insight into the mechanism of function of the trifunctionalized nanoengagers, correlative studies were conducted using the A431 tumor model. A biodistribution study via an ex vivo near-infrared fluorescence imaging method indicated that the tumor took up an insignificant amount (<0.2%ID/g) of Cy5-labeled α-CD16/α-4-1BB NPs when co-administered with α-EGFR (FIGS. 10F and 11A). On the other hand, approximately 5.7±1.3%ID/g of the administered Cy5-labeled α-EGFR/α-CD16/α-4-1BB NPs accumulated in the tumor, about three times (p=0.0251) higher than that of Cy5-labeled α-EGFR NPs plus Cy5-labeled α-CD16/α-4-1BB NPs. The increased tumor uptake of EGFR-targeted trivalent NPs facilitated the engagement of circulating NK cells with tumor cells and increased the number of tumor-infiltrating NK1.1⁺ NK cells by about 17-fold (FIG. 11B), but no significant DNA damage was observed (FIG. 11C), as indicated in histopathologic studies. Since the NP-co-anchored α-CD16 and α-4-1BB also effectively activated the NK cells, the serum cytokine levels (e.g., TNF-α, INF-γ) significantly increased after treatment with the trivalent nanoengagers (FIG. 11D). Notably, these enhancements can only be observed in mice treated with the EGFR-targeted nanoengagers but not in the combination of α-EGFR NPs and α-CD16/α-4-1BB NPs (FIG. 11A). This is because NK cell activation by the α-CD16/α-4-1BB NPs did not facilitate tumor cell recognition by NK cells, thus leading to ineffective immune activation. A similar biodistribution trend was observed in the chemoimmunotherapy groups (FIGS. 11A and 11E). All EPI-encapsulated NPs functionalized with α-EGFR have significantly higher EPI uptake (8.5-10%ID/g) compared to free EPI (≈3%ID/g). The increased EPI uptake is consistent with the higher γ-H2AX expression (leading to DNA damage), as observed in the histopathological study (FIG. 11C). Neither the administration of α-EGFR/α-CD16/α-4-1BB EPI NPs nor α-EGFR/α-CD16/α-4-1BB NPs plus free EPI affected the serum cytokine levels, suggesting that the concurrent EPI treatment did not affect NK cell antitumor activity (FIG. 11D). This comprehensive mechanistic study confirmed that the tailor-made EPI-encapsulated nanoengagers can effectively deliver cytotoxic chemotherapeutics to cancer cells and facilitate NK cells to attack the tumor cells.

This study presents a new translatable multimodal cancer treatment platform for the concurrent targeted delivery of chemotherapeutics and activating the host's innate immune system to eradicate cancer. It demonstrated that EGFR-targeted trivalent nanoengagers of the present invention can recruit and activate circulating NK cells to attack tumor cells while simultaneously delivering a therapeutic dose of cytotoxic chemotherapeutics to the tumor cells. Comprehensive in vitro and in vivo studies demonstrated that this synthetic lethality cannot be achieved by conventional chemoimmunotherapy strategies. The data demonstrated that both robust NK activation and biological targeting are critical in NK cell-mediated cancer treatments, and NP-based treatments are uniquely suited for this application. The need for biological targeting also suggests that systemic/non-specific toxicity is low with this approach. The simple modular design of nanoengagers allows an easy exchange of chemotherapeutics, targets moieties for the treatment of a different type of cancer and engages with various types of immune cells. The development of nanoengager platforms could improve currently available combination immunotherapy strategies.

The foregoing is illustrative of the present invention, and is not to be construed as limiting thereof. The invention is defined by the following claims, with equivalents of the claims to be included therein.

### TABLES

TABLE 1
Summary of physiochemical properties of drug-free and
EPI-encapsulated antibody-functionalized nanoparticles.

| | | | Mean | | |
|---|---|---|---|---|---|
| | | | intensity- | | |
| | Mean | Modal | average | | |
| | number- | number- | diameter,^{#} nm | Mean zeta | Antibody |
| | average | average | (mean | potential in | grafting density, |
| | diameter | diameter | polydispersity | 0.1M | μg antibody/mg |
| | (mean D_{N}),\hat{ } nm | (modal D_{N}),\hat{ } nm | index, PDI) | PBS,* mV | NPs |
| Drug-free azide- | 73 ± 3 | 68 | 98 ± 3 | −1.23 ± 0.48 | / |
| functionalized | | | (0.152) | | |
| PEG-PLGA NPs | | | | | |
| EPI NPs | 91 ± 4 | 80 | 110 ± 7 | −1.48 ± 0.71 | / |
| | | | (0.153) | | |
| Drug-free α-CD16 | 95 ± 4 | 96 | 121 ± 9 | −1.01 ± 0.52 | 100 μg antibody/6 |
| NPs | | | (0.219) | | mg NPs = 16.6 μg |
| | | | | | antibody/mg NPs |
| Drug-free α-4-1BB | 99 ± 5 | 93 | 118 ± 4 | − 1.16 ± 0.64 | 100 μg antibody/6 |
| NPs | | | (0.231) | | mg NPs = 16.6 μg |
| | | | | | antibody/mg NPs |
| Drug-free α- | 110 ± 6 | 98 | 127 ± 6 | −1.01 ± 0.91 | (100 + 100) |
| CD16/α-4-1BB | | | (0.274) | | μg antibody/6 |
| NPs | | | | | mg NPs = 33.3 μg |
| | | | | | antibody/mg NPs |
| Drug-free α-EGFR | 95 ± 3 | 88 | 110 ± 2 | −1.46 ± 0.43 | 100 μg antibody/6 |
| NPs | | | (0.191) | | mg NPs = 16.6 μg |
| | | | | | antibody/mg NPs |
| Drug-free α- | 110 ± 6 | 99 | 126 ± 8 | −0.93 ± 0.61 | (100 + 100 + 100) |
| EGFR/α-CD16/α- | | | (0.280) | | μg antibody per 6 |
| 4-1BB NPs | | | | | mg NPs = 50 μg |
| | | | | | antibody/mg NPs |
| α-EGFR EPI NPs | 95 ± 4 | 90 | 121 ± 5 | −1.10 ± 0.95 | 100 μg antibody/6 |
| | | | (0.184) | | mg NPs = 16.6 μg |
| | | | | | antibody/mg NPs |
| α-EGFR/α- | 112 ± 7 | 104 | 134 ± 7 | −0.74 ± 0.97 | (100 + 100 + 100) |
| CD16/α-4-1BB | | | (0.301) | | μg antibody per 6 |
| EPI NPs | | | | | mg NPs = 50 μg |
| | | | | | antibody/mg NPs |
| α-EGFR | / | / | 14 ± 1 | −0.76 ± 0.87 | / |
| α-CD16 | / | / | 15 ± 2 | −0.85 ± 1.02 | / |
| α-4-1BB | / | / | 12 ± 1 | −0.51 ± 0.43 | / |

1. A nanoparticle comprising:
at least two first targeting agents that bind a first target or two different first targets on a natural killer (NK) cell surface; and
at least one second targeting agent that binds a second target on a cancer cell surface, wherein the second targeting agent is different from the first targeting agent.

2. (canceled)

3. The nanoparticle of claim 1, wherein the at least two first targeting agents bind two different targets on the NK cell surface.

4. The nanoparticle of claim 1, wherein the first targets on the NK cell surface [[is]]are selected from the group consisting of CD16, 4-1BB, NKG2D, TRAIL, NKG2C, CD137, OX40, CD27, KIRs, NKG2a, dnam-1, 2b4, NKp30a, NKp30b, NKp30c, antibody Fc component, and any combination thereof.

5. The nanoparticle of claim 4, wherein the two different first targets on the NK cell surface are CD16 and 4-1BB.

6. The nanoparticle of claim 1, wherein the cancer cell is a cell from a adenocarcinoma, thymoma, sarcoma, brain cancer, head and neck cancer, esophageal cancer, gastric cancer, lung cancer, bladder cancer, kidney cancer, liver cancer, pancreatic cancer, uterine cancer, ovarian cancer, cervical cancer, anal cancer, melanoma, prostate cancer, breast cancer, blood cell cancer, colorectal cancer, or any combination thereof.

7. The nanoparticle of claim 1, wherein the second target on the cancer cell surface is selected from the group consisting of EGFR, PSMA, Nectin-4, mucin, HER-2, CD30, CD22, and any combination thereof.

8. The nanoparticle of claim 7, wherein the second target on the cancer cell surface is EGFR.

9. The nanoparticle of claim 1, wherein the first targeting agent and/or the second targeting agent is an antibody or active fragment thereof.

10. The nanoparticle of claim 9, wherein the antibody or active fragment thereof is selected from the group consisting of a monoclonal antibody, a Fab fragment, a Fab'-SH fragment, a FV fragment, a scFV fragment, a (Fab′)₂ fragment, an Fc-fusion protein, and any combination thereof.

11. The nanoparticle of claim 1, further comprising a therapeutic agent.

12. The nanoparticle of claim 11, wherein the therapeutic agent is a chemotherapeutic agent selected from the group consisting of epirubicin (EPI), doxorubicin, cisplatin, oxaliplatin, carboplatin, daunorubicin, taxol, docetaxel, gemcitabine, 5-fluorouracil, mitomycin, cytarabine, cytoxan, and any combination thereof.

13. The nanoparticle of claim 12, wherein the chemotherapeutic agent is epirubicin (EPI).

14-16. (canceled)

17. A method of inducing cytotoxicity in a cancer cell, comprising contacting the cancer cell with the nanoparticle of claim 1 14 under conditions whereby the second targeting agent binds the second target on the surface of the cancer cell.

18. The method of claim 17, wherein the nanoparticle further comprises a therapeutic agent.

19-25. (canceled)

26. A method of treating cancer in a subject in need thereof, comprising administering to the subject an effective amount of the nanoparticle of claim 1 under conditions whereby the two first targeting agents bind the two different first targets on the surface of the NK cell and whereby the second agent binds the second target on the surface of the cancer cell.

27-29. (canceled)

30. The method of claim 26, further comprising the step of administering to the subject an effective amount of a therapeutic agent and/or radiation therapy.

31. The method of claim 30, wherein the therapeutic agent is a chemotherapeutic agent selected from the group consisting of epirubicin (EPI), doxorubicin, cisplatin, oxaliplatin, carboplatin, daunorubicin, taxol, docetaxel, gemcitabine, 5-fluorouracil, mitomycin, cytarabine, cytoxan, and any combination thereof.

32. The method of claim 31, wherein the chemotherapeutic agent is epirubicin (EPI).

33. The method of claim 26, wherein the subject is a mammal.

34. The method of claim 33, wherein the mammal is a human.

35-40. (canceled)
